(19) United States
(12) Patent Application Publication
(10) Pub. No.: US 2025/0002450 A1
(43) Pub. Date: Jan. 2, 2025

(54) CATIONIC STEROID COMPOUNDS, METHOD OF OBTAINING THEREOF, FORMULATIONS COMPRISING THEREOF AND THEIR USES

(71) Applicants: UNIVERSIDADE DO PORTO, PORTO (PT); CIIMAR & CENTRO INTERDISCIPLINAR DE INVESTIGACÃO MARINHA E AMBIENTAL, MATOSINHOS (PT); FACULDADE DE CIÊNCIAS DA UNIVERSIDADE DE LISBOA, LISBOA (PT)

(72) Inventors: Maria Emília DA SILVA PEREIRA DE SOUSA, PORTO (PT); Ana Rita DA CONCEIÇÃO NEVES, PORTO (PT); Marta RAMOS PINTO CORREIA DA SILVA CARVALHO GUERRA, PORTO (PT); Joana Manuela MACHADO FREITAS DA SILVA, MATOSINHOS (PT); Fernando André PEREIRA MARQUES DURÃES, PORTO (PT); Paulo Manuel RODRIGUES MARTINS DA COSTA, PORTO (PT); Maria Eugénia RIBEIRO PINTO, PORTO (PT); Elisabete RIBEIRO SILVA GERALDES, LISBOA (PT); Filipe José MENEZES MERGULHÃO, PORTO (PT); Marisa Da Conceição LIMA GOMES, PORTO (PT); Rita Daniela TEIXEIRA DOS SANTOS, PORTO (PT)

(21) Appl. No.: 18/717,702

(22) PCT Filed: Dec. 11, 2022
(86) PCT No.: PCT/IB2022/062027

§ 371 Date: Jun. 7, 2024

(30) Foreign Application Priority Data
Dec. 10, 2021 (PT) 117633

(51) Int. Cl. C07C 237/52; A61K 31/165; A61K 31/4184; A61K 31/4453; A61K 31/497; A61K 31/5377; A61P 31/04; A61P 31/10; C07D 211/14; C07D 235/14; C07D 241/04; C07D 265/30

(52) U.S. Cl. CPC C07C 237/52; A61K 31/165; A61K 31/4184; A61K 31/4453; A61K 31/497; A61K 31/5377; A61P 31/04; A61P 31/10; C07D 211/14; C07D 235/14; C07D 241/04; C07D 265/30

(57) ABSTRACT

The present invention relates to cationic steroid compounds and methods of obtaining thereof. The present invention further relates to the incorporation of such compounds in a polymeric matrix composition or a coating composition, as well as their use as antimicrobials.

The present invention claims the benefit of and priority to the Portuguese provisional patent application no. 117633, filed on Dec. 10, 2021, the entire contents thereof being herein incorporated by reference.

### TECHNICAL FIELD

The present invention relates to cationic steroid compounds and methods of obtaining thereof. The present invention further relates to the incorporation of such compounds in a polymeric matrix composition or a coating composition, as well as their use as antimicrobials.

### BACKGROUND ART

Currently, multidrug-resistant (MDR) infections are one of the most worrisome threats, driving the search for new antimicrobials compounds. In 2015, in Europe, around 670000 infections were caused by antibiotic-resistant pathogens and 33000 deaths resulted from antibiotic-resistant infections.

In contrast to Gram-positive bacteria, the control of the Gram-negative bacteria growth is challenging due to the low permeability of the outer membrane. Important antibiotic classes currently in use are not able to efficiently penetrate the outer membrane and therefore are ineffective against Gram-negative bacteria. Therapeutic choices for fungal diseases are also limited, particularly for invasive infections, and resistance has been described for all antifungal agents, including for Candida species.

Cationic peptide antibiotics (CPAs), such as polymyxin B (FIG. 1A), are facially amphiphilic natural peptides highly effective against drug-resistant Gram-negative pathogens. The synthetic complexity and the hemolytic activity of CPAs have hampered their development as effective antibiotics.

Cationic steroid antibiotics (CSAs), also known as ceragenins, have proven antibacterial activities similar to many CPAs. CSAs are smaller molecules, easier to synthesize, and/or modify than CPAs. CSAs mimic the required morphology of CPAs, combining a bile acid scaffold and amine groups attached. The antibacterial activity of CSAs arises from a bactericidal effect. Many CSAs are broad-spectrum bactericidal agents, active against both Gram-negative and Gram-positive bacteria, and can permeabilize the outer membrane of Gram-negative bacteria. In Gram-negative bacteria, CSAs target the lipid A portion of lipopolysaccharides (LPS). Additionally, CSAs have shown activity against MDR bacteria. The bactericidal properties of CSAs are due to membrane disruption, and a moderate degree of selectivity for prokaryotic over eukaryotic membranes can be observed.

Particularly, a study showed that a specific CSA, CSA-13 (FIG. 1B), had a low potential to develop resistance in Pseudomonas aeruginosa and Acinetobacter baumannii, compared with ciprofloxacin and colistin. CSAs have also been described as fungicidal against several pathogenic fungal species, including Candida spp. For instance, this activity has been associated with the incorporation of CSAs in the lipophilic environment of the Candida membrane, resulting in morphological changes in membrane structure and leading to cell death. This activity was extended to drug-resistant fungi in both planktonic states and biofilms.

Bile acid derivatives have attracted attention due to their steroid scaffold. In fact, this scaffold has been proven to possess gelator properties [1-3]. Furthermore, these compounds have been previously studied as antimicrobial agents, with several research groups synthesizing mainly derivatives of cholic and deoxycholic acid [4-11]. Noteworthy are deoxycholic amides, that have proven to display a broad spectrum concerning antimicrobial activity, being promising both against Gram-positive and Gram-negative bacteria, as well as fungi [12-16]. For example, U.S. Pat. No. 5,583,239 A described a series of deoxycholic acid derivatives as antimicrobials. However, different substitution patterns were used, wherein only aliphatic non-cyclic diamines were used to prepare amides.

Several documents of the state of the art have disclosed CSAs and their use. For example, document US 2021363174 A1 discloses CSA compounds having endogenous groups based on natural terpenes, amino acids, and cholic acid or derivative of cholic acid, and methods of manufacturing CSA compounds having endogenous groups.

As can be seen in document US 2017258963 A1, CSAs can be incorporated into compositions to provide effective antimicrobial, anti-inflammatory, analgesic, anti-swelling and/or tissue-healing properties, wherein the CSA compounds are mixed with a biologically compatible material so that the CSA compounds are incorporated within the composition, forming a reservoir of CSA compounds within the resulting bolus of the treatment composition after injection and/or application.

Urinary tract infections (UTIs) are one of the most common infections and in the case of chronic and recurrent UTIs, the major challenge is the eradication of microbial biofilm, which considerably increases bacterial resistance to antimicrobial agents. Recently, a study was performed to understand the potential of ceragenins in conjunction with the antimicrobial LL-37 peptide against multi-drug resistant Escherichia coli, responsible for about 80% of all UTIs. Biofilms are sessile communities of microbial cells that are attached to a surface due to the production of a matrix of extracellular polymeric substances (EPS). These structures protect from external factors such as antimicrobial drugs, making biofilm-associated infections particularly difficult to treat. Biofilms have an important role in the progression of UTIs but are particularly relevant after catheterization and stenting.

As disclosed in document US 2018272034 A1, the CSA compounds can be incorporated into medical implants to provide effective antimicrobial properties, more specifically, to prevent microbial fouling caused by bacterial and/or fungal biofilms.

In recent research performed by the group of inventors of the present invention, a bile acid derivative with a primary amine at C-24, compound 1 (FIG. 1C), was recently found to be active against the growth of marine biofilm-forming bacterial strains [17].

The nature of the group extending from the C-24 position has been shown in previous studies to influence the bactericidal activity of the ceragenins against Gram-negative bacteria, such as E. coli. This Enterobacteriaceae species is the most frequent etiological agent of UTIs, nonetheless, Pseudomonas spp., Enterococcus spp., Staphylococcus spp. are also frequently isolated. Multidrug-resistance plays a key role in the successful treatment and prevention of recurring UTIs, being E. coli extended-spectrum β-lactamase (ESBL)-producing strains of particular importance. Besides community-acquired UTIs, these microbial species are also known to be associated with hospital-acquired UTIs, including catheter and stent-associated urinary tract infections (CAUTIs). Salmonella enterica serovar Typhimurium is one of the most frequent serotypes responsible for animal and human infections in different regions of the world, and its treatment has been affected by the emergence of resistance. Yersinia ruckeri and Listonella anguillarum are causative agents of severe diseases on fish farms all over the world, being responsible for severe economic losses worldwide.

Immobilization of antimicrobials on medical devices rather than coating them on the surface reduces their amount required to achieve the antimicrobial effect as well as prolongs their activity. Several antimicrobial surfaces have been described. However, many of these compounds are associated with anaphylaxis, cytotoxicity or low efficiency. These limiting aspects prompt the use of antibiotics, through substance-releasing coating and substance covalent immobilization. The release strategy offers the potential for extended activity but failed to achieve delivery of a sustained and effective dosage over a relatively prolonged period. Covalent attachment of drugs to the implant surface aims to achieve long-lasting antibacterial activity. However, the effectiveness of coatings with classical antibiotics is strongly dependent on the spectrum of activity of the chosen drug, and the possibility of development of antimicrobial resistance in a relatively short time. Therefore, alternative answers must be developed [18].

Nonetheless, in the covalent attachment approach when an active molecule is modified using a crosslinker agent, it is essential that the pharmacophoric groups responsible for the antimicrobial activity remain unaltered.

These facts are disclosed in order to illustrate the technical problem addressed by the present disclosure.

### SUMMARY OF INVENTION

1. In a first aspect, the present invention discloses a compound of general formula (I)

[symbol]

(I)
or an acceptable salt, a hydrate, a solvate, an enantiomer, an atropisomer, a polymorph or an ester thereof
wherein
X is an atom selected from the group consisting of N, O or C, with the proviso that X is bonded to at least one of R₁ or R₂ when X is N or C, and X is optionally bonded to R₁ or R₂ when X is O;
each of R₁ or R₂ are independently selected from the group consisting of H, a C₁-C₈ alkyl group, a C₆-C₁₂ aryl group, a 3-12-membered heterocyclyl ring, a 5-12 membered heteroaryl ring, —NH—COOR3 wherein R3 is H or a C1-C8 alkyl group, —CO—NH2, —NHCOCF3, —NH-Aryl, —NH—C(Aryl)3, —N═CH-Aryl, —NH—S(O)2-Aryl wherein Aryl is benzyl or toluyl; a —COO-alkyl group, —NH₂, —NH—,

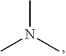

### —N═, or

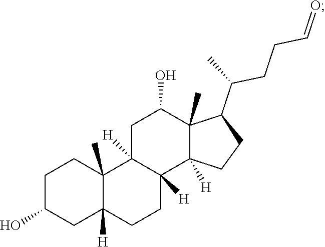 [SMILES: C[C@H](CCC=O)[C@H]1CC[C@H]2[C@@H]3CC[C@@H]4C[C@H](O)CC[C@]4(C)[C@H]3C[C@H](O)[C@]12C]

with the proviso that when R₁ or R₂ is —NH—,

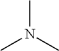

or —N═, X is taken together with the R₁ and R₂ of the compound of general formula (I) to form a 3-12-membered heterocyclyl or condensed heterocyclyl ring or 5-12 membered heteroaryl or condensed heteroaryl ring, provided that the rules of valency permit, wherein each heterocyclyl or heteroaryl ring optionally contains at least one additional heteroatom selected from the group consisting of O and N;
R₄ is selected from the group consisting of H, a single bond or a double bond;
with the proviso when R₄ is a single bond or a double bond, R₄ is taken together with X to form a 3-12-membered heterocyclyl ring or 5-12 membered heteroaryl ring;
n is an integer in the range from 1 to 6.

In a second aspect, the present invention discloses the methods for obtaining the compounds of the present invention.

In a third aspect, the present invention discloses a composition comprising at least one of the compounds herein disclosed and a pharmaceutically acceptable excipient, wherein the composition is a polymeric matrix composition (which is used to obtain articles such as medical devices) or a coating composition.

In a fourth aspect, the present invention discloses the use of the compounds of the present invention, or the compositions comprising thereof, as a medicament in humans or animals to treat or prevent bacterial or fungal infections, as well as its medical use for antibiofilm purposes in medical devices, such as ureteral stents.

### Technical Problem

Urinary tract infections (UTIs) are one of the most common infections and in the case of chronic and recurrent UTIs, the major challenge is the eradication of microbial biofilm, which considerably increases bacterial resistance to antimicrobial agents.

### Solution to Problem

The present invention relates to cationic steroid compounds and methods of obtaining thereof. The present invention further relates to the incorporation of such compounds in a polymeric matrix composition or a coating composition, as well as their use as antimicrobials.

### Advantageous Effects of Invention

The compounds described herein can be applied as broad antimicrobials in priority infections in humans against ESKAPE bacteria and also against microorganisms responsible for infections in aquaculture.

These results indicate that the incorporation of the compounds of the present invention into PDMS coating matrix, especially compound 1, significantly reduced the E. coli biofilm formation. Nonetheless, with this covalent attachment approach using a crosslinker agent, it would be expected the loss of activity due to masking groups responsible for the antimicrobial activity. Immobilization of antimicrobials on medical devices rather than coating them on the surface reduces their amount required to achieve the antimicrobial effect as well as prolongs their activity.

Additional features and advantages will be set forth in part in the description that follows, and in part will be obvious from the description, or may be learned by practice of the embodiments disclosed herein. It is to be understood that both the foregoing brief summary of the invention and the following detailed description of the embodiments are exemplary and not restrictive of the embodiments disclosed herein or as claimed.

### BRIEF DESCRIPTION OF DRAWINGS

In order to promote an understanding of the principles according to the modalities of the present invention, reference will be made to the modalities illustrated in the figures and the language used to describe them.

It should also be understood that there is no intention to limit the scope of the invention to the content of the figures and that modifications to the inventive features illustrated herein, as well as additional applications of the principles and embodiments illustrated, which would normally occur to a person skilled in the art having the possession of this description, are considered within the scope of the claimed invention.

FIG. 1

FIG. 1 shows the chemical structure of (A) polymyxin B, (B) CSA-13 and (C) compound 1 of the present invention, a derivative previously reported as an antifouling agent.

FIG. 2

FIG. 2A-I shows the chemical structure of deoxycholic amide compounds 1 to 9 synthesized according to the present invention.

FIG. 3

FIG. 3 graphically shows a time-kill plot of E. coli ATCC 25922 treated with 64-8 g/mL of compound 1, showing a concentration-dependent killing. Data presented are means of three independent experiments.

FIG. 4

FIG. 4 graphically shows the effect of compound 1 on membrane integrity of E. coli ATCC 25922 after 0, 1, and 18 h of exposure, as expressed by SYTO® 9/PI ratio. Controls used included exposure to colistin (COL; 8 and 1 μg/mL) and AMP (4 μg/mL). Data are shown as mean±standard error of mean (SEM) of three independent assays. Values significantly different from untreated control: ****p<0.0001 (Two-way ANOVA followed by Dunnett's test).

FIG. 5

FIG. 5 graphically shows the effect of compound 1 on the metabolic activity of E. coli ATCC 25922 after 0, 1, and 18 h of exposure, as expressed by % of untreated control. Controls used included exposure to COL (8 and 1 μg/mL) and AMP (4 μg/mL). Data are shown as mean±SEM of three independent assays. Values significantly different from untreated control: ****p<0.0001 (Two-way ANOVA followed by Dunnett's test).

FIG. 6

FIG. 6 graphically shows the percentage of biofilm formation of E. coli ATCC 25922 (A), P. aeruginosa ATCC 27853 (B), S. aureus ATCC 29213 (C) and E. faecalis ATCC 29212 (D) after 24 h incubation with compound 1. Data are shown as mean±SEM of three independent assays. Values significantly different from untreated control: *p<0.05, **p<0.01, ***p<0.001, ****p<0.0001 (One-way ANOVA followed by Dunnett's test).

FIG. 7

FIG. 7 graphically shows the number of total (▪) and culturable (▪) E. coli cells on PDMS (control) and compound 1-M films after 48 h of biofilm formation in dynamic conditions. Data are shown as mean±standard deviation (SD) of two independent experiments performed in triplicate. Statistical comparisons were performed using Mann-Whitney test: *** p<0.001 indicates significant differences from the control or between different compound 1 contents.

FIG. 8

FIG. 8 graphically shows the time-kill plot of Candida albicans ATCC 10231 treated with 64-8 μg/mL of compound 1, showing a concentration-dependent killing effect. Data presented are means of at least three independent experiments.

FIG. 9

FIG. 9 graphically shows the effect of compound 1 on membrane integrity of C. albicans ATCC 10231 after 5 min exposure, as expressed by SYTO® 9/PI ratio. Controls used included exposure to amphotericin B (AMB; 8 μg/mL), fluconazole (FLC; 8 μg/mL) and sodium azide (10 mM) for 5 min and exposure of an untreated control to 80 degrees C. for 20 min. Data are shown as mean±SEM of three independent assays. Values significantly different from untreated control: *p<0.05, ***p<0.001, (One-way ANOVA followed by Dunnett's test).

FIG. 10

FIG. 10 graphically shows the effect of compound 1 on membrane integrity of C. albicans ATCC 10231 after 5 min exposure, as expressed by % of extracellular K+. Controls used included exposure to amphotericin B (AMB; 8 μg/mL), fluconazole (FLC; 8 μg/mL) and sodium azide (10 mM) for 5 min and exposure of an untreated control to 80 degrees C. for 20 min. Data are shown as mean±SEM of three independent assays. Values significantly different from untreated control: ***p<0.001, ****p<0.0001 (One-way ANOVA followed by Dunnett's test).

FIG. 11

FIG. 11 graphically shows the effect of compound 1 on the activity of mitochondrial dehydrogenases of C. albicans ATCC 10231 after 2 h of exposure, as expressed by % of mitochondrial activity. Controls used included exposure to amphotericin B (AMB; 8 g/mL), fluconazole (FLC; 8 μg/mL), voriconazole (VRC; 8 μg/mL) and sodium azide (10 mM) for 2 h and exposure of an untreated control to 80 degrees C. for 20 min. Data are shown as mean±SEM of three independent assays. Values significantly different from untreated control: ***p<0.001, ****p<0.0001 (One-way ANOVA followed by Dunnett's test).

FIG. 12

FIG. 12 graphically shows the percentage of Candida albicans ATCC 10231 biofilm formation after 48 h incubation with compound 1 (A); percentage of C. albicans ATCC 10231 germ tube formation at 3 h incubation with compound 1 (B). Data are shown as mean±SEM of three independent assays. Values significantly different from untreated control: *p<0.05, **p<0.01, ****p<0.0001 (One-way ANOVA followed by Dunnett's test).

FIG. 13

FIG. 13 shows (A) the synthetic route for the synthesis of compounds 2-8 and (B) the synthetic route for the synthesis of compound 9.

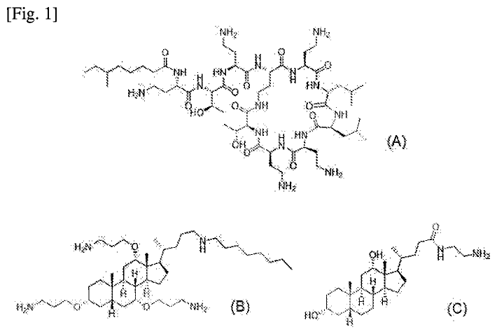

### DESCRIPTION OF EMBODIMENTS

The present invention discloses new cationic steroid compounds presenting antibacterial activity and/or antifungal activity and methods of obtaining thereof. Furthermore, the present invention relates to the incorporation of such compounds in a polymeric matrix composition or a coating composition, as well as their use as antimicrobials.

More specifically, the present invention relates, in a first aspect, to a compound of general formula (I)

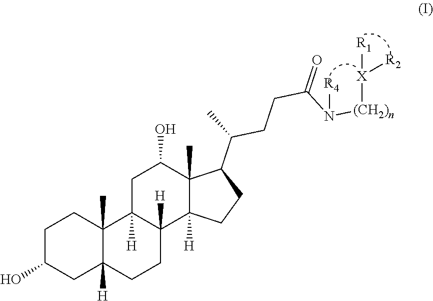

or an acceptable salt, a hydrate, a solvate, an enantiomer, an atropisomer, a polymorph or an ester thereof
wherein
X is an atom selected from the group consisting of N, O or C, with the proviso that X is bonded to at least one of R₁ or R₂ when X is N or C, and X is optionally bonded to R₁ or R₂ when X is O;
each of R₁ or R₂ are independently selected from the group consisting of H, a C₁-C₈ alkyl group, a C₆-C₁₂ aryl group, a 3-12-membered heterocyclyl ring, a 5-12 membered heteroaryl ring, —NH—COOR3 wherein R3 is H or a C₁-C₈ alkyl group, —CO—NH₂, —NHCOCF₃, —NH-Aryl, —NH—C(Aryl)₃, —N═CH-Aryl, —NH—S(O)₂-Aryl wherein Aryl is benzyl or toluyl; a —COO-alkyl group, —NH₂, —NH—,

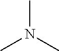

### —N═, or

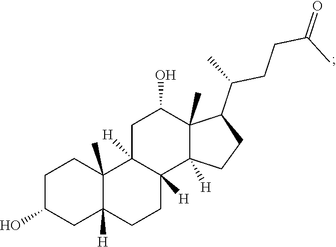 [SMILES: CC(=O)CC[C@@H](C)[C@H]1CC[C@H]2[C@@H]3CC[C@@H]4C[C@H](O)CC[C@]4(C)[C@H]3C[C@H](O)[C@]12C]

with the proviso that when R₁ or R₂ is —NH—,

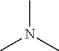

or —N═, X is taken together with the R₁ and R₂ of the compound of general formula (I) to form a 3-12-membered heterocyclyl or condensed heterocyclyl ring or 5-12 membered heteroaryl or condensed heteroaryl ring, provided that the rules of valency permit, wherein each heterocyclyl or heteroaryl ring optionally contains at least one additional heteroatom selected from the group consisting of 0 and N;
R₄ is selected from the group consisting of H, a single bond or a double bond;
with the proviso when R₄ is a single bond or a double bond, R₄ is taken together with X to form a 3-12-membered heterocyclyl ring or 5-12 membered heteroaryl ring;
n is an integer in the range from 1 to 6.

In an embodiment of the present invention, the term “alkyl”, by itself or as part of another substituent, e.g., alkoxy, haloalkyl or aminoalkyl, means, unless otherwise stated, a saturated hydrocarbon radical having the number of carbon atoms designated (i.e. C₁-C₆ means one, two, three, four, five or six carbons) and includes straight, branched chain, cyclic and polycyclic groups. Examples include: methyl, ethyl, propyl, isopropyl, butyl, isobutyl, tert-butyl, pentyl, neopentyl, hexyl, cyclohexyl, norbornyl and cyclopropylmethyl.

“Substituted alkyl” means alkyl, as defined above, substituted by one, two or three substituents preferably independently selected from the group consisting of halogen, —OH, —O(C₁-C₄) alkyl, —NH₂, —N(CH₃)₂, —CO₂H, —CO₂(C₁-C₄)alkyl, —CF₃, —CONH₂, —SO₂NH₂, —C(—NH)NH₂, —CN and —NO₂. More preferably, the substituted alkyl contains one or two substituents independently selected from halogen, —OH, NH₂, —N(CH₃)₂, trifluoromethyl and —CO2H; most preferably, independently selected from halogen and —OH. Examples of substituted alkyls include, but are not limited to, 2,2-difluoropropyl, 2-carboxycyclopentyl and 3-chloropropyl.

In an embodiment of the present invention, the term “aryl” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl. Preferred are phenyl and naphthyl, most preferred is phenyl.

In an embodiment of the present invention, the term “heteroalkyl” by itself or in combination with another term means, unless otherwise stated, a stable straight or branched chain radical consisting of the stated number of carbon atoms and one or two heteroatoms selected from the group consisting of O, N, and S, and wherein, in the sulfur heteroatoms may be optionally oxidized and the nitrogen heteroatoms may be optionally quaternized or oxidized. The oxygens bonded to oxidized sulfur or nitrogen may be present in addition to the one or two heteroatoms in the heteroalkyl group. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—SO₂—NH—CH₃, —CH₂—S—CH₂—CH₃, and —CH₂CH₂—S(—O)—CH₃. Up to two heteroatoms may be consecutive, such as, for example, —CH₂—NH—OCH₃, or —CH₂—CH₂—S—S—CH₃.

In an embodiment of the present invention, the term “heterocycle” or “heterocyclyl” or “heterocyclic” by itself or as part of another substituent means, unless otherwise stated, an unsubstituted or substituted, stable, mono- or multicyclic heterocyclic ring system which consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom which affords a stable structure.

In an embodiment of the present invention, the term “heteroaryl” or “heteroaromatic” refers to a heterocycle having aromatic character. A monocyclic heteroaryl group is a 5-, 6-, or 7-membered ring, examples of which are pyrrolyl, furyl, thienyl, pyridyl, pyrimidinyl and pyrazinyl. A polycyclic heteroaryl may comprise multiple aromatic rings or may include one or more rings which are partially saturated. Examples of polycyclic heteroaryl groups containing a partially saturated ring include tetrahydroquinolyl and 2,3-dihydrobenzofuryl. For compounds of Formula I, the attachment point on ring Q is understood to be on an atom which is part of an aromatic monocyclic ring or a ring component of a polycyclic aromatic which is itself an aromatic ring. The attachment point on ring Q may be a ring carbon or a ring nitrogen and includes attachment to form aromatic quaternary ammonium salts such as pyridinium.

Examples of non-aromatic heterocycles include monocyclic groups such as: aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include: pyridyl, pyrazinyl, pyrimidinyl, particularly 2- and 4-pyrimidyl, pyridazinyl, thienyl, furyl, pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1,8-naphthyridinyl, 1,4-benzodioxanyl, coumarin, dihydrocoumarin, benzofuryl, particularly 3-, 4-, 1,5-naphthyridinyl, 5-, 6- and 7-benzofuryl, 2,3-dihydrobenzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl, and quinolizidinyl.

In an embodiment of the present invention, the term “amine” or “amino” refers to radicals of the general formula —NRR′, wherein R and R′ are independently selected from hydrogen or a hydrocarbyl radical, or wherein R and R′ combined form a heterocycle, Examples of amino groups include: —NH2, methyl amino, diethyl amino, anilino, benzyl amino, piperidinyl, piperazinyl and indolinyl.

In an embodiment of the present invention, the carbamate include, without limitation, fluorenylmethyl carbamate, t-butyl carbamate, benzyl carbamate, methyl carbamate, ethyl carbamate, 2,2,2-trichloroethyl carbamate, 2-(trimethylsilyl)ethyl carbamate, 1,1-dimethyl-2,2,2-trichloroethyl carbamate, p-methoxybenzyl carbamate, pnitrobenzylcarbamate, p-bromobenzyl carbamate, p-chlorobenzyl carbamate, and 2,4-dichlorobenzyl carbamate, preferably t-butyl carbamate.

In a preferred embodiment of the present invention, in the compound of formula (I): X is N or C, wherein X is bonded to R1 and R2; each of R1 or R2 are independently selected from the group consisting of H, a C₁-C₈ alkyl group, a C₆-C₁₂ aryl group or a 5-12 membered heteroaryl ring, the C₁-C₈ alkyl group being ethyl, the C₆-C₁₂ aryl group being phenyl, the 5-12 membered heteroaryl ring being benzoimidazolyl, R₄ is H; and n is an integer in the range from 2 to 4.

In another preferred embodiment of the present invention, in the compound of formula (I): X is an atom selected from the group consisting of N, O or C, with the proviso that: when X is N, R₄ is a single bond; and R₄ is taken together with X to form a piperidyl or a piperazinyl, which are optionally substituted by a —COO—C₁-C₈ alkyl; and when X is O, R₄ is a single bond; and R₄ is taken together with X to form a morpholinyl; and when X is C, X is bonded to R₁ and R₂, which are H; and R₄ is a single bond; and R₄ is taken together with X to form a piperidyl; and n is an integer in the range from 2 to 4.

In another preferred embodiment of the present invention, in the compound of formula (I): X is N and is bonded to R1 and R2; each of R1 or R2 are independently selected from the group consisting of H, a C₁-C₈ alkyl group, or

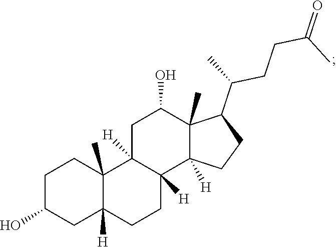 [SMILES: CC(=O)CC[C@@H](C)[C@H]1CC[C@H]2[C@@H]3CC[C@@H]4C[C@H](O)CC[C@]4(C)[C@H]3C[C@H](O)[C@]12C]

R₄ is a single bond; and R₄ is taken together with X to form a piperidyl or a piperazinyl; and n is an integer in the range from 2 to 4.

In a preferred embodiment of the present invention, the salt is a fluoride, chloride, bromide, iodide, acetate, citrate, maleate, or mesylate.

In a more preferred embodiment of the invention, the compound is one of the following:

### Compound 1

(R)—N-(2-aminoethyl)-4-((3R,5R,8R,9S,10S,12S,13R,14S,17R)-3,12-dihydroxy-10,13-dimethylhexadecahydro-1H-cyclopenta[a]phenanthren-17-yl) pentanamida

### Compound 2

(R)-4-((3R,5R,8R,9S,10S,12S,13R,14S,17R)-3,12-dihydroxy-10,13-dimethylhexadecahydro-1H-cyclopenta[a]phenanthren-17-yl)-1-(piperidin-1-yl) pentan-1-one

### Compound 3

(R)-4-((3R,5R,8R,9S,10S,12S,13R,14S,17R)-3,12-dihydroxy-10,13-dimethylhexadecahydro-1H-cyclopenta[a]phenanthren-17-yl)-1-morpholinopentan-1-one

### Compound 4

(R)-4-((3R,5R,8R,9S,10S,12S,13R,14S,17R)-3,12-dihydroxy-10,13-dimethylhexadecahydro-1H-cyclopenta[a]phenanthren-17-yl)-1-(4-((S)-4-((3S,5S,8S,9R,10R,12R,13S,14R,17S)-3,12-dihydroxy-10,13-dimethylhexadecahydro-1H-cyclopenta[a]phenanthren-17-yl)pentanoyl)piperazin-1-yl) pentan-1-one

### Compound 5

tert-butyl 4-((R)-4-((3R,5R,8R,9S,10S,12S,13R,14S,17R)-3,12-dihydroxy-10,13-dimethylhexadecahydro-1H-cyclopenta[a]phenanthren-17-yl)pentanoyl)piperazine-1-carboxylate

### Compound 6

(R)—N-(2-(diethylamino)ethyl)-4-((3R,5R,8R,9S,10S,12S,13R,14S,17R)-3,12-dihydroxy-10,13-dimethylhexadecahydro-1H-cyclopenta[a]phenanthren-17-yl)pentanamide

### Compound 7

(R)—N-(2-(1H-benzo[d]imidazol-2-yl)ethyl)-4-((3R,5R,8R,9S,10S,12S,13R,14S,17R)-3,12-dihydroxy-10,13-dimethylhexadecahydro-1H-cyclopenta[a]phenanthren-17-yl) pentanamide

### Compound 8

(R)-4-((3R,5R,8R,9S,10S,12S,13R,14S,17R)-3,12-dihydroxy-10,13-dimethylhexadecahydro-1H-cyclopenta[a]phenanthren-17-yl)-N-(2-(phenylamino)ethyl)pentanamide

### Compound 9

(R)-4-((3R,5R,8R,9S,10S,12S,13R,14S,17R)-3,12-dihydroxy-10,13-dimethylhexadecahydro-1H-cyclopenta[a]phenanthren-17-yl)-1-(piperazin-1-yl) pentan-1-one

In a second aspect, the present invention discloses a method for obtaining the compounds of the present invention, wherein two approaches are used:
(i) the coupling of an amine, preferably N,N-diisopropylethylamine, and a carboxylic acid of formula (II)

[symbol]

(II)
using (1-cyano-2-ethoxy-2-oxoethylidenaminooxy) dimethylaminomorpholino-carbenium hexafluorophosphate (COMU) as coupling agent; and using (1-cyano-2-ethoxy-2-oxoethylidenaminooxy) dimethylamino-morpholino-carbenium hexafluorophosphate (COMU).
(ii) the deprotection of an amide of formula (III)

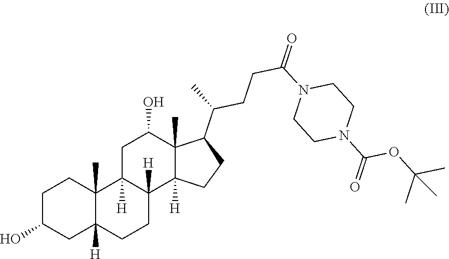

with trifluoroacetic acid.

In a third aspect, the present invention also relates to a composition comprising at least one of the compounds 1 to 9 herein disclosed and a pharmaceutically acceptable excipient, wherein the composition is a polymeric matrix composition (which is used to obtain articles such as medical devices) or a coating composition.

In a preferred embodiment of the invention, the composition comprises 0.1 to 10 wt % of the compound of the present invention, preferably from 0.5 to 1.5 wt % of the compound of the present disclosure.

In a preferred embodiment of the invention, the polymeric matrix composition is a polydimethylsiloxane (PDMS) based matrix composition. PDMS is one of the most widely used materials for medical devices, for example, for the constructions of urinary tract devices.

In an embodiment of the invention, the above-mentioned composition may further comprise an antibiotic, wherein the antibiotic is a fluoroquinolone selected from the group consisting of ciprofloxacin, norfloxacin, pefloxacin, enofloxacin, ofloxacin, levofloxacin, moxifloxacin, nalidixic acid or mixtures thereof; a macrolide selected from the group consisting of erythromycin, azithromycin, or mixtures thereof; an aminoglycoside, preferably gentamicin; a β-lactam selected from the group consisting of cefoxitin, cefotaxime, ampicillin, cephalothin, or mixtures thereof; a polypeptide selected from the group consisting of polymyxin B; vancomycin; rifampicin; trimethoprim-sulfamethoxazole or mixtures thereof.

In a preferred embodiment of the invention, the composition further comprises at least one of the additives selected from the group consisting of: a dye, a polymer, a filler, an essential oil, a stabilizer, a surfactant, a crosslinker agent, a curing agent, a biocide, a solvent, or mixtures thereof.

In a preferred embodiment of the present invention, the dye is selected from at least one of the group consisting of azo-, phthalocyanine and anthraquinone derivatives, titanium dioxide (titanium (IV) oxide), calcium carbonate, iron oxides (black, yellow and red), zinc oxide and carbon black.

In a preferred embodiment of the present invention, the polymer is selected from at least one of the group consisting of polyacrylic, polyvinyl acrylic or polystyrene acrylic, polydimethylsiloxane (PDMS) or polyurethane.

In a preferred embodiment of the present invention, the filler is selected from at least one of the group consisting of talc, silica, kaolin, clay or calcium carbonate.

In a preferred embodiment of the present invention, the essential oil is selected from at least one of the group consisting of linseed oil, tung oil, and soya oil.

In a preferred embodiment of the present invention, the stabilizer is selected from at least one of the group consisting of UV stabilizers, hindered amine light stabilizers.

In a preferred embodiment of the present invention, the surfactant is selected from at least one of the group consisting of siloxane, polyoxyethylene glycol octylphenol ethers, dioctyl sodium sulfosuccinate.

In a preferred embodiment of the present invention, the crosslinker agent is a solvent-based 3-glycidyloxypropyl) trimethoxysilane (GLYMO) epoxy silane crosslinker.

In a preferred embodiment of the present invention, the curing agent comprises epoxy or hydroxy functional groups.

In a preferred embodiment of the present invention, the biocide is selected from at least one of the group consisting of cuprous oxide, copper pyrithione, zinc pyrithione, zineb, cuprous thiocyanate, dichlorooctylisothiazolinone (DCOIT), Irgarol, pyridinetriphenylborane (PTPB), diuron, tralopyril and dichlofluanid.

In a preferred embodiment of the present invention, the solvent is selected from at least one of the group consisting of oxygenated solvents, hydrocarbons or halogenated solvents. More preferably, the solvent is selected from at least one of the group consisting of ethanol, ethyl acetate, methyl ethyl ketone, xylene, toluene, acetone, or isophorone.

In a fourth aspect of the present invention, it is disclosed the use of the compounds of the present invention, or the compositions comprising thereof, as a medicament in humans or animals to treat or prevent bacterial or fungal infections, as well as its medical use for antibiofilm purposes in medical devices.

In an embodiment of the invention, any of the compounds herein disclosed except compound 3 are for use in the treatment of Gram-positive bacterial infections, preferably caused by Staphylococcus spp. and/or Enterococcus spp., more preferably caused by Staphylococcus aureus and/or Enterococcus faecalis. In a preferred embodiment of the invention, compounds 5, 7 and 8 are preferably used in the treatment of bacterial infections caused by Staphylococcus aureus. In another preferred embodiment of the invention, compounds 1, 2, 4, 6 and 9 are preferably used in the treatment of bacterial infections caused by Enterococcus faecalis, the compound 1 being the most preferably used.

In an embodiment of the invention, compounds 1 and 2 herein disclosed are for use in the treatment of Gram-positive bacterial infections, preferably caused by Streptococcus spp., more preferably Streptococcus pyogenes, the compound 2 being the most preferably used.

In an embodiment of the invention, compounds 1 or 9 herein disclosed are for use in the treatment of Gram-negative bacterial infections, preferably caused by Escherichia spp., more preferably caused by E. coli, the compound 1 being the most preferably used.

In an embodiment of the invention, compounds 1, 8, or 9 herein disclosed are for use in the treatment of Gram-negative bacterial infections, preferably caused by Salmonella spp., more preferably caused by Salmonella enterica serovar Typhimurium, the compound 1 being the most preferably used.

In an embodiment of the invention, compounds 1, 6, 8, or 9 herein disclosed are for use in the treatment of Gram-negative bacterial infections, preferably caused by Acinetobacter spp., more preferably caused by A. baumannii.

In an embodiment of the invention, compounds 1, 4, 6, or 9 herein disclosed are for use in the treatment of Gram-negative bacterial infections, preferably caused by Listonella spp., more preferably caused by Listonella anguilarum, the compound 1 being the most preferably used.

In an embodiment of the invention, compounds 1 and 4 herein disclosed are for use in the treatment of Gram-negative bacterial infections, preferably caused by Yersinia spp., more preferably caused by Yersinia ruckeri, the compound 1 being the most preferably used.

In an embodiment of the invention, all the compounds herein disclosed except compound 3 are for use in the treatment of Gram-negative bacterial infections, preferably caused by Tenacibaculum spp., more preferably caused by Tenacibaculum maritimum, the compound 2 being the most preferably used.

In an embodiment of the invention, compounds 1 and 2 herein disclosed are for use in the treatment of Gram-negative bacterial infections, preferably caused by Campylobacter jejuni, the compound 1 being the most preferably used.

In an embodiment of the invention, compound 1 herein disclosed are for use in the treatment of Gram-negative bacterial infections, preferably by Klebsiella spp. and/or Pseudomonas spp., more preferably caused by Klebsiella pneumoniae and/or P. aeruginosa.

The practice of the invention is illustrated by the following non-limiting examples.

### EXAMPLES

### Methods for Obtaining the Compounds of the Invention

In the present invention, all reagents used were from analytical grade. Deoxycholic acid (II), morpholine, N,N-diisopropylethylamine, piperidine, (1-cyano-2-ethoxy-2-oxoethylidenaminooxy)dimethylamino-morpholino-carbenium hexafluorophosphate, N,N-diethylethylenediamine, 2-(1H-benzimidazole-2-yl)ethylamine, trigluoroacetic acid (TFA), and N-phenylethyldiamine were purchased from Sigma (Sigma-Aldrich Co. Ltd., Gillinghan, UK). PiperazineBoc was purchased from TCI (Tokyo Chemical Industry Co. Ltd., Chuo-ku, Tokyo, Japan). Column chromatography purifications were performed using flash silica Merck 60, 230-400 mesh (EMD Millipore Corporation, Billerica, MA, USA). Melting points were measured in a Köfler microscope and are uncorrected. Infrared spectra were recorded in a KBr microplate in a FTIR spectrometer Nicolet iS10 from Thermo Scientific (Waltham, MA, USA) with Smart OMNI-Transmission accessory (Software 188 OMNIC 8.3). ¹H and ¹³C NMR spectra were recorded in CDCl₃ (Deutero GmbH, Kastellaun, Germany) or DMSO-d₆ (Sigma-Aldrich Chemie GmbH, Steinheim, Germany) at room temperature unless otherwise mentioned on Bruker AMC instrument (Bruker Biosciences Corporation, Billerica, MA, USA), operating at 300 MHz for ¹H and 75 MHz for ¹³C, or Bruker Avance III (Bruker Biosciences Corporation, Billerica, MA, USA), operating at 400 MHz for ¹H and 100 MHz for ¹³C). Carbons were assigned according to HSQC and or HMBC experiments. High-resolution mass spectra (HRMS) were measured on a Bruker FTMS APEX III mass spectrometer (Bruker Corporation, Billerica, MA, USA) recorded as ESI (Electrospray) made in Centro de Apoio Cientifico e Tecnolòxico á Investigation (CACTI, University of Vigo, Pontevendra, Spain), or on a LTQ Orbitrap XL hybrid mass spectrometer (Thermo Fischer Scientific, Bremen, Germany) at CEMUP, University of Porto, Portugal.

### Statistical Analysis

GraphPad Prism 6 for Windows (GraphPad Software, San Diego, CA, USA) was used to perform all statistical calculations. Three tests were performed to check the normality of the data distribution: Kolmogorov-Smirnov, D'Agostino & Pearson omnibus, and Shapiro-Wilk normality tests.

For data with parametric distribution, One-way ANOVA was used to perform the statistical comparisons, followed by Dunnett's multiple comparisons test. The Mann-Whitney test and Kruskal-Wallis nonparametric test followed by Dunn's multiple comparisons test were used to perform the statistical comparisons for data with nonparametric distribution.

Data of three independent experiments are presented as mean±standard error of the mean (SEM). Levels of statistical significance *p<0.05, **p<0.01, ***p<0.001 and ****p<0.0001 were used.

Details of the performed statistical analysis are described in each figure legend. Differences were considered to be significant at p values lower than 0.05.

### Obtainment of the Compounds 1 to 8

Compounds 1 to 8 were obtained according to the reaction shown in FIG. 13A. The chemistry of compound 1 is described in reference [18].

Compounds 2 to 8 were synthesized by the coupling of an amine and a carboxylic acid, using (1-cyano-2-ethoxy-2-oxoethylidenaminooxy) dimethylaminomorpholino-carbenium hexafluorophosphate (COMU) as coupling agent. N,N-Diisopropylethylamine was preferably used as the hindered base, and dichloromethane as solvent.

In the present invention, the general conditions for the synthesis of compounds 2 to 8 are as follows. Deoxycholic acid (II) (0.250 g, 0.6 mmol) was suspended in dichloromethane (CH₂Cl₂, 5 mL). N,N-Diisopropylethylamine was added dropwise (1.3 mmol, 2 eq.) until dissolution. The reaction was cooled to 0 degrees C., and (1-cyano-2-ethoxy-2-oxoethylidenaminooxy) dimethylamino-morpholino-carbenium hexafluorophosphate (COMU, 1.3 mmol, 2 eq.) was added and stirred for 30 min. The appropriate amine was then added, and the reaction was gradually heated to room temperature and occurred overnight. The products of the reaction were first extracted with an aqueous solution of hydrochloric acid (1 M), and the aqueous layer was alkalinized with a saturated solution of sodium bicarbonate until basic pH, followed by extraction with CH₂Cl₂. The organic layers were concentrated via rotary evaporator and, in the case of compounds 2 to 6 the crude product was purified by flash chromatography using chloroform:methanol (9:1) as a mobile phase. In the case of compounds 7-8, the crude product was purified by washing with CH₂Cl₂ and/or crystallization, yielding compounds 2-8. All the reactions performed were analyzed by thin-layer chromatography (TLC), using the mobile phase chloroform:methanol:triethylamine (8:2:0.1), and the stationary phase precoated plates with 0.2 mm of thickness using Merck silica gel 60 (GF₂₅₄). A 20% solution of sulfuric acid in methanol was used as a visualization reagent.

In an embodiment, the characterization of (R)-4-((3R,5R,8R,9S,10S,12S,13R,14S,17R)-3,12-dihydroxy-10,13-dimethylhexadecahydro-1H-cyclopenta[a]phenanthren-17-yl)-1-(piperidin-1-yl)pentan-1-one (compound 2) is as follows: Yield: 244.8 mg, 88% as white solid; mp 78.1-79.8 degrees C.; IR vₘₐₓ (KBr): 3423, 2935, 2861, 1753, 1739, 1694, 1627, 1606, 1458, 1373, 1307, 1255, 1223, 1192, 1136, 1094, 1066, 1044, 1014, 969, 943, 919, 755, 668 cm⁻¹; ¹H NMR (CDCl₃, 300.13 MHz) δ (ppm): 3.98 (1H, t, J=3.03 Hz), 3.61 (1H, m), 2.39 (1H, m), 2.24 (1H, m), 2.17 (1H, d, J=4.89 Hz), 1.81 (8H, m), 1.64 (8H, m), 1.52 (8H, m), 1.40 (5H, m), 1.29 (2H, m), 1.24 (2H, m), 1.09 (2H, m), 0.99 (3H, d, J=6.36 Hz), 0.90 (3H, s), 0.68 (3H, s); ¹³C NMR (CDCl₃, 75.48 MHz) δ (ppm): 172.1, 73.3, 72.0, 48.4, 47.4, 46.6, 42.8, 42.2, 36.6, 36.2, 35.5, 35.3, 34.2, 33.8, 31.6, 30.6, 30.4, 29.8, 28.8, 27.6, 27.3, 26.3, 25.6, 24.7, 23.8, 23.3, 17.7, 12.9, 11.4; HRMS (ESI⁺): m/z [C₂₉H₄₉NO₃+H]⁺ calcd. for [C₂₉H₅₀NO₃]: 460.3785; found 460.3779.

In an embodiment, the characterization of (R)-4-((3R,5R,8R,9S,10S,12S,13R,14S,17R)-3,12-dihydroxy-10,13-dimethylhexadecahydro-1H-cyclopenta[a]phenanthren-17-yl)-1-morpholinopentan-1-one (compound 3) is as follows: Yield: 227.2 mg, 82% as white solid; mp 166.0-167.8° C.; IR vₘₐₓ (KBr): 3495, 3388, 2988, 2922, 2857, 1621, 1479, 1466, 1440, 1391, 1372, 1361, 1302, 1277, 1253, 1111, 1095, 1063, 1047, 1014, 966, 580 cm⁻¹; ¹H NMR (CDCl₃, 300.13 MHz) δ (ppm): 3.98 (1H, t, J=3.02 Hz), 3.63 (7H, m), 3.46 (2H, m), 2.37 (1H, m), 2.21 (1H, m), 1.80 (8H, m), 1.62 (5H, s), 1.52 (4H, m), 1.38 (6H, m), 1.24 (1H, s), 1.10 (2H, m), 0.99 (3H, d, J=6.33 Hz), 0.90 (3H, s), 0.68 (3H, s); ¹³C NMR (CDCl₃, 75.48 MHz) δ (ppm): 172.5, 73.3, 71.9, 67.1, 66.8, 48.4, 47.3, 46.6, 46.2, 42.2, 42.0, 36.5, 36.1, 35.4, 35.3, 34.2, 33.8, 31.3, 30.6, 30.0, 28.8, 27.6, 27.2, 26.2, 23.8, 23.3, 17.7, 12.9; HRMS (ESI⁺): m/z [C₂₈H₄₇NO₄+H]⁺ calcd. for [C₂₈H₄₈NO₄]: 462.3578; found 462.3577.

In an embodiment, the characterization of (R)-4-((3R,5R,8R,9S,10S,12S,13R,14S,17R)-3,12-dihydroxy-10,13-dimethylhexadecahydro-1H-cyclopenta[a]phenanthren-17-yl)-1-(4-((S)-4-((3S,5S,8S,9R,10R,12R,13S,14R,17S)-3,12-dihydroxy-10,13-dimethylhexa decahydro-1H-cyclopenta[a]phenanthren-17-yl)pentanoyl)piperazin-1-yl)pentan-1-one (compound 4) is as follows: Yield: 159.3 mg, 32% as white solid; mp 237.5-238.4° C.; IR vₘₐₓ (KBr): 3423, 2921, 2861, 1560, 1468, 1446, 1375, 1255, 1051 cm⁻¹; ¹H NMR (CDCl₃, 500.16 MHz) δ (ppm): 3.98 (2H, t, J=3.2 Hz), 3.61 (6H, m), 3.46 (4H, m), 2.40 (2H, m), 2.25 (2H, m), 1.80 (28H, m), 1.52 (10H, m), 1.41 (9H, m), 1.26 (5H, m), 1.00 (6H, d, J=6.4 Hz), 0.91 (6H, s), 0.68 (6H, s); ¹³C NMR (CDCl₃, 125.77 MHz) δ (ppm): 172.6, 73.3, 72.0, 48.4, 47.3, 46.6, 45.5, 42.2, 41.7, 36.5, 36.1, 35.4, 34.2, 33.8, 31.3, 30.6, 29.8, 28.8, 27.7, 27.2, 26.2, 23.8, 23.3, 17.7, 17.6, 12.9; HRMS (ESI⁺): m/z [C₅₂H₈₆N₂O₆+H]⁺ calcd. for [C₅₂H₈₇N₂O₆]: 835.6559; found 835.6566.

In an embodiment, the characterization of tert-butyl 4-((R)-4-((3R,5R,8R,9S,10S,12S,13R,14S,17R)-3,12-dihydroxy-10,13-dimethylhexadecahydro-1H-cyclopenta[a]phenanthren-17-yl)pentanoyl)piperazine-1-carboxylate (compound 5) is as follows: Yield: 221.2 mg, 66% as white solid; mp 100.3-101.7° C.; IR vₘₐₓ (KBr): 3442, 2935, 2863, 1700, 1633, 1459, 1421, 1366, 1286, 1252, 1169, 1129, 1090, 1045, 997 cm⁻¹; ¹H NMR (CDCl₃, 300.13 MHz) δ (ppm): 3.98 (1H, t, J=2.99 Hz), 3.61 (3H, m), 3.40 (7H, m), 2.39 (1H, m), 2.23 (1H, m), 1.79 (8H, m), 1.59 (4H, s), 1.52 (5H, m), 1.47 (10H, s), 1.27 (4H, m), 1.09 (3H, m), 0.99 (3H, d, J=6.3 Hz), 0.90 (3H, s), 0.68 (3H, s); 13C NMR (CDCl₃, 75.48 MHz) δ (ppm): 172.4, 154.7, 80.4, 73.3, 71.9, 48.4, 47.3, 46.6, 45.6, 42.2, 41.5, 36.6, 36.2, 35.4, 35.3, 34.2, 33.8, 31.4, 30.6, 30.3, 28.8, 28.5, 27.7, 27.2, 26.2, 23.8, 23.3, 17.7. 12.9; HRMS (ESI⁺): m/z [C₃₃H₅₆N₂O₅+H]⁺ calcd. for [C₃₃H₅₇N₂O₅]: 561.4267; found 561.4258.

In an embodiment, the characterization of (R)—N-(2-(diethylamino)ethyl)-4-((3R,5R,8R,9S,10S,12S,13R,14S,17R)-3,12-dihydroxy-10,13-dimethylhexadecahydro-1H-cyclopenta[a]phenanthren-17-yl)pentanamide (compound 6) is as follows: Yield: 262.8 mg, 89% as white solid. mp 95.5-96.4° C.; IR vₘₐₓ (KBr): 3404, 2937, 2858, 1625, 1560, 1467, 1446, 1385, 1375, 1090, 1045, 850, 559 cm⁻¹; ¹H NMR (CDCl₃, 300.13 MHz) δ (ppm): 7.63 (1H, s), 3.96 (1H, t, J=2.97 Hz), 3.57 (3H, m), 3.00 (7H, m), 2.32 (1H, m), 2.17 (1H, m), 1.77 (10H, m), 1.47 (13H, m), 1.29 (8H, t, J=7.23 Hz), 0.99 (3H, d, J=5.97 Hz), 0.89 (3H, s), 0.66 (3H, s); 13C NMR (CDCl₃, 75.48 MHz) δ (ppm): 175.3, 73.2, 71.8, 52.7, 48.4, 47.5, 46.9, 46.7, 42.2, 36.5, 36.1, 35.6, 35.5, 35.4, 34.3, 33.7, 33.1, 31.5, 30.6, 28.7, 27.7, 27.3, 26.3, 23.9, 23.3, 17.6, 12.8, 9.6; HRMS (ESI⁺): m/z [C₃₀H₅₄N₂O₃+H]⁺ calcd. for [C₃₀H₅₅N₂O₃]: 491.4212; found 491.4206.

In an embodiment, the characterization of (R)—N-(2-(1H-benzo[d]imidazol-2-yl)ethyl)-4-((3R,5R,8R,9S,10S,12S,13R,14S,17R)-3,12-dihydroxy-10,13-di methylhexadecahydro-1H-cyclopenta[a]phenanthren-17-yl)pentanamide (compound 7) is as follows: Yield: 83.0 mg, 26% as white solid; mp 152.7-154.6 degrees C.; IR vₘₐₓ (KBr): 3411, 3096, 2926, 2861, 1671, 1525, 1448, 1416, 1369, 1308, 1273, 1252, 1225, 1087, 1052, 1014, 769, 754, 736 cm⁻¹; ¹H NMR (DMSO-d₆, 300.13 MHz) δ (ppm): 12.30 (1H, s), 7.93 (1H, t, J=4.26 Hz), 7.46 (2H, m), 7.10 (2H, dd, J=2.34 and 6.45 Hz), 4.44 (1H, d, J=3.15 Hz), 4.15 (1H, d, J=3.09 Hz), 3.77 (1H, d, J=2.76 Hz), 3.48 (2H, q, J=5.45 Hz), 2.93 (2H, t, J=5.45 Hz), 2.08 (1H, m), 1.84 (6H, m), 1.55 (7H, m), 1.25 (12H, m), 0.89 (3H, d, J=4.80 Hz), 0.84 (3H, s), 0.57 (3H, s); ¹³C NMR (DMSO-d₆, 75.48 MHz) δ (ppm): 172.8, 152.8, 110.7, 79.3, 79.1, 78.9, 78.6, 71.0, 69.9, 47.4, 46.2, 45.9, 41.6, 37.2, 36.3, 35.6, 35.1, 35.0, 33.8, 32.9, 32.5, 31.6, 30.6, 30.2, 29.0, 28.6, 27.1, 26.7, 26.1, 23.5, 23.1, 17.1, 12.4; HRMS (ESI⁺): m/z [C₃₃H₄₉N₃O₃+H]⁺ calcd. for [C₃₃H₅₀N₃O₃]: 536.3852; found 536.3843.

In an embodiment, the characterization of (R)-4-((3R,5R,8R,9S,10S,12S,13R,14S,17R)-3,12-dihydroxy-10,13-dimethylhexadecahydro-1H-cyclopenta[a]phenanthren-17-yl)-N-(2-(phenylamino)ethyl)pentanamide (compound 8) is as follows: Yield: 128.3 mg, 42% as white solid; mp 180.1-181.9 degrees C.; IR vₘₐₓ (KBr): 3615, 3293, 3083, 3019, 2931, 2864, 1659, 1605, 1553, 1513, 1499, 1447, 1377, 1335, 1298, 1233, 1194, 1152, 1114, 1083, 1063, 1043, 1013, 747, 693, 604 cm⁻¹; ¹H NMR (DMSO-d₆, 400.14 MHz) δ (ppm): 8.30 (1H, d, J=2.64 Hz), 7.87 (1H, dd, J=4.86 and 8.12 Hz), 7.06 (2H, td, J=2.39 and 7.94 Hz), 6.54 (2H, m), 5.54 (1H, m), 4.45 (1H, t, J=3.38 Hz), 4.17 (1H, t, J=3.18 Hz), 3.78 (1H, t, J=3.36 Hz), 3.20 (2H, m), 3.04 (2H, m), 1.93 (7H, m), 1.56 (7H, m), 1.26 (11H, m), 1.03 (2H, m), 0.92 (3H, d, J=4.12 Hz), 0.84 (3H, s), 0.58 (3H, s); ¹³C NMR (DMSO-d₆, 100.63 MHz) δ (ppm): 172.9, 148.6, 128.9, 115.6, 111.9, 79.2, 71.0, 69.9, 47.4, 46.2, 46.0, 42.6, 41.6, 38.0, 36.3, 35.6, 35.1, 35.0, 33.8, 32.9, 32.5, 31.6, 30.2, 28.6, 27.2, 27.0, 26.1, 23.5, 23.1, 17.1, 12.4; HRMS (ESI⁺): m/z [C₃₂H₅₀N₂O₃+H]⁺ calcd. for [C₃₂H₅₁N₂O₃]: 511.3900; found 511.3913.

### Obtainment of the Compounds 9

Compound 9 was obtained by the deprotection of deoxycholic amide (III), using trifluoroacetic acid, as show in FIG. 13B. Dichloromethane (CH₂Cl₂) was used as solvent.

In the present disclosure, the general conditions for the synthesis of compound 9 are as follows. To a solution of compound tert-butyl 4-((R)-4-((3R,5R,8R,9S,10S,12S,13R,14S,17R)-3,12-dihydroxy-10,13-dimethylhexadecahydro-1H-cyclopenta[a]phenanthren-17-yl)pentanoyl)piperazine-1-carboxylate (III) (100.3 mg, 0.18 mmol) in CH₂Cl₂, 1.58 mL of trifluoroacetic acid (TFA, 114 eq.) was added dropwise, and the reaction was stirred at room temperature for 2 h. The reaction was quenched with saturated sodium bicarbonate and extracted with CH₂Cl₂. The crude product obtained after solvent evaporation was washed with methanol, furbishing compound 9. The reaction was analyzed by thin-layer chromatography (TLC), using the mobile phase chloroform:methanol:triethylamine (8:2:0.1), and the stationary phase precoated plates with 0.2 mm of thickness using Merck silica gel 60 (GF₂₅₄). A 20% solution of sulfuric acid in methanol was used as a visualization reagent.

In an embodiment, the characterization of (R)-4-((3R,5R,8R,9S,10S,12S,13R,14S,17R)-3,12-dihydroxy-10,13-dimethylhexadecahydro-1H-cyclopenta[a]phenanthren-17-yl)-1-(piperazin-1-yl)pentan-1-one (compound 9) is as follows: Yield: 37 mg, 45% as white solid. mp 229.3-230.8 degrees C. (methanol). IR vₘₐₓ (KBr) 3417, 2936, 2862, 1602, 1470, 1445, 1254, 1052 cm⁻¹; ¹H NMR (CDCl₃, DMSO-d₆, 400.14 MHz) δ (ppm): 7.62 (1H, m), 3.93 (1H, m), 3.49 (5H, m), 3.37 (1H, m), 2.86 (12H, m), 2.60 (1H, m), 2.38 (1H, m), 2.22 (1H, m), 1.81 (7H, m), 1.60 (4H, m), 1.26 (4H, m), 1.12 (1H, m), 1.00 (3H, d, J=4.36 Hz), 0.89 (3H, s), 0.66 (3H, s); ¹³C NMR (CDCl₃, DMSO-d₆, 100.63 MHz) δ (ppm): 171.4, 71.7, 70.3, 47.3, 46.2, 46.1, 45.7, 45.7, 45.1, 41.9, 41.4, 35.8, 35.3, 34.8, 34.7, 33.5, 32.8, 30.7, 29.7, 29.5, 28.1, 26.9, 26.6, 25.5, 23.1, 22.6, 16.7, 12.0; HRMS (ESI⁺): m/z [C₂₈H₄₈N₂O₃+H]⁺ calcd. for [C₂₈H₄₉N₂O₃]: 461.3743; found 461.3747.

### Antibacterial Activity

The present invention further relates to the antibacterial activity of the compounds herein disclosed.

Twelve reference bacterial strains and nine MDR clinically relevant bacterial strains were used in this study. Gram-negative bacteria comprised reference strains E. coli ATCC 25922, K. pneumoniae ATCC 13883, Salmonella enterica serovar Typhimurium CECT 443, P. aeruginosa ATCC 27853, A. baumannii ATCC 19606, C. jejuni ATCC 33560 (ATCC—American Type Culture Collection; CECT—Colección Espanola de Cultivos Tipo); clinical isolates E. coli SA/2, an extended-spectrum β-lactamase (ESBL)-producing strain and P. aeruginosa 33b, a pan-drug-resistant isolate; and animal isolates C. jejuni P5/4, a ciprofloxacin-resistant strain, C. jejuni 4432, C. jejuni 4433, C. jejuni 4448, and E. coli 2252, a COL-resistant strain [42]. Gram-positive strains included S. aureus ATCC 29213, E. faecalis ATCC 29212, S. pyogenes ATCC 19615, and environmental isolates methicillin-resistant S. aureus (MRSA) 66/1 [43], and VAN-resistant enterococci (VRE) E. faecalis B3/101 [44]. Gram-negative fish pathogens Yersinia ruckeri ATCC 29473, Listonella anguillarum ATCC 1924, and Tenacibaculum maritimum ATCC 43397 were also included.

Strains were kept in Trypto-Casein Soy agar (TSA—Biokar Diagnostics, Allone, Beauvais, France) slants and, before each assay, were sub-cultured in Mueller-Hinton agar (MHA—Biokar Diagnostics, Allone, Beauvais, France). Y. ruckeri ATCC 29473 and L. anguillarum ATCC 1924 were kept in Nutrient Agar (Condalab, Madrid, Spain) and T. maritimum ATCC 43397 was kept in TMM agar (Condalab, Madrid, Spain), and were sub-cultured in the respective culture media before each assay. For S. pyogenes ATCC 19615, CAMHB was supplemented with 3.75% lysed horse blood (LBH—Oxoid, Basingstoke, England), whereas for C. jejuni it was supplemented with 2.5% LHB. Colony-forming unit counts of the inoculum were conducted to determine the initial inoculum size (which should be approximately 5×10⁵ CFU/mL). Sterility and growth controls were included in each assay. The 96-well U-shaped untreated polystyrene microtiter plates were incubated for 20 h at 37 degrees C. (42 degrees C. for C. jejuni, in a microaerophilic atmosphere) and the minimal inhibitory concentration (MIC) was determined as the lowest concentration of compound that prevented visible growth. The minimal bactericidal concentration (MBC) was determined by spreading 100 μL of the content of the wells with no visible growth on MH plates. The MBC was determined as the lowest concentration of compound that killed 99.9% of the initial inoculum after overnight incubation at 37 degrees C. Two independent assays were performed for reference and MDR strains.

An initial screening of the antibacterial activity of the compounds was performed by the Kirby-Bauer disk diffusion method, as recommended by the Clinical and Laboratory Standards Institute (CLSI). Briefly, sterile 6 mm blank paper disks (Oxoid, Basingstoke, England) impregnated with 15 μg of each compound were placed on inoculated MH agar plates. A blank disk with dimethylsulfoxide (DMSO) was used as a negative control. MH inoculated plates were incubated for 18-20 h at 37 degrees C. At the end of incubation, the inhibition halos where measured. The MIC was used to determine the antibacterial activity of each compound, in accordance with the recommendations of the CLSI. Two-fold serial dilutions of the compounds were prepared in Mueller-Hinton Broth 2 (MHB2—Sigma-Aldrich, St. Louis, MO, USA) within the concentration range of 0.062-64 μg/mL. CTX ranging between 0.031-16 μg/mL was used as a control. Sterility and growth controls were included in each assay. Purity checks and colony counts of the inoculum suspensions were also performed in order to ensure that the final inoculum density closely approximates the intended number (5×10⁵ CFU/mL). The MIC was determined as the lowest concentration of compound that inhibited growth of the bacteria. The MBC was assessed by spreading 10 μL of culture collected from wells showing no visible growth on MH agar plates. The MBC was determined as the lowest concentration at which no colonies grew after 16-18 h incubation at 37 degrees C. These assays were performed in duplicate.

In order to evaluate the combined effect of the compounds and clinically relevant antimicrobial drugs, a screening was conducted using the disk diffusion method, as previously described. A set of antibiotic disks (Oxoid, Basingstoke, England) to which the isolates were clinically resistant was selected: CTX (30 μg) for ESBL producing E. coli SA/2, OXA (1 μg) for S. aureus 66/1, and VAN (30 μg) for E. faecalis B3/101. Antibiotic disks alone (controls) and antibiotic disks impregnated with 15 μg of each compound were placed on MH agar plates seeded with the respective bacteria. Sterile 6 mm blank papers impregnated with 15 μg of each compound alone were also tested. A blank disk with DMSO was used as a negative control. MH inoculated plates were incubated for 18-20 h at 37 degrees C. Potential synergism was recorded when the halo of an antibiotic disk impregnated with a compound was greater than the halo of the antibiotic or compound-impregnated blank disk alone.

Synergy with Other Antimicrobial Drugs

The combined effect of compound 1 and CTX for E. coli SA/2, VAN for E. faecalis B3/101, COL for E. coli 2252, erythromycin for E. coli ATCC 25922, and OXA for S. aureus 66/1 was evaluated by the checkerboard method. No synergistic interactions were detected. Potential synergy was also evaluated with COL for E. coli 2252, a strain whose COL resistance is conferred by a mobile resistance gene (mcr-1), a fractional inhibitory concentration index (FICI) of 0.6 was obtained, which falls in the ‘no interaction’ category (0.5<FICI≤4, ‘no interaction’), suggesting an additive effect. The interaction between compound 1 and erythromycin was assessed for E. coli ATCC 25922. Gram-negative bacteria are intrinsically resistant to erythromycin, a macrolide that inhibits protein synthesis, and cannot penetrate the outer membrane of Gram-negative bacteria, which is impermeable to hydrophobic antibiotics. Ascertain ceragenins have been previously described to be able to enhance the efficacy of such antibiotics, this possibility was investigated for compound 1 and a FICI of 0.6 was obtained. Even though this result is classified as ‘no interaction’, (‘synergy’ corresponds to FICI≤0.5), it is relevant to note that 16 μg/mL of compound 1 (½×MIC) lowered the erythromycin MIC from 16 to 0.125 μg/mL.

### Antibacterial Synergy Testing

The potential synergy between compound 1 and clinically relevant antimicrobial drugs was screened using the Kirby-Bauer method, as previously described. A set of antibiotic discs (Oxoid, Basingstoke, England) to which the isolates were resistant was selected: CTX (30 μg) for E. coli SA/2, VAN (30 μg) for E. faecalis B3/101, and OXA (1 μg) for S. aureus 66/1. Antibiotic discs impregnated with 15 μg of each compound were placed on seeded MH plates. The controls used included antibiotic discs alone, blank paper discs impregnated with 15 μg of each compound alone, and blank discs impregnated with DMSO. Plates with CTX were incubated for 18-20 h and plates with VAN and OXA were incubated for 24 h at 37 degrees C. [45]. Potential synergy was considered when the inhibition halo of an antibiotic disc impregnated with the compound was greater than the inhibition halo of the antibiotic or compound-impregnated blank disc alone.

The antibacterial activity of the nine compounds herein described were evaluated for several bacterial species that included Gram-negative and Gram-positive bacteria. The compounds revealed a broad spectrum of activity, preferably compound 1, showing activity against all microorganisms tested (Table 1), including reference strains and MDR isolates. This antibacterial effect was bactericidal, with MBC being equal to or two-fold higher than the respective MIC.

TABLE 1
Antibacterial activity of the deoxycholic amides 1 to 9 against reference and clinical bacterial strains.
MIC and MBC are expressed in μg/mL and disk diffusion in mm.

| | 1 | | | | | | | | | | |
|---|---|---|---|---|---|---|---|---|---|---|---|
| | | | Disk | 2 | | 3 | | 4 | | 5 | |
| Strains | MIC | MBC | diffusion | MIC | MBC | MIC | MBC | MIC | MBC | MIC | MBC |
| Escherichia | 32 | 64 | 8 | >64 | >64 | >64 | >64 | >64 | >64 | >64 | >64 |
| coli ATCC | | | | | | | | | | | |
| 25922 | | | | | | | | | | | |
| E. coli SA/2 | 32 | 32 | 8 | >64 | >64 | >64 | >64 | >64 | >64 | >64 | >64 |
| (ESBL) | | | | | | | | | | | |
| E. coli 2252 | 64 | ND | ND | >64 | >64 | >64 | >64 | >64 | >64 | >64 | >64 |
| (COL-resistant) | | | | | | | | | | | |
| Klebsiella | 64 | 64 | ND | >64 | >64 | >64 | >64 | >64 | >64 | >64 | >64 |
| pneumoniae | | | | | | | | | | | |
| ATCC 13883 | | | | | | | | | | | |
| Salmonella | 16 | 16 | ND | >64 | >64 | >64 | >64 | 64 | 64 | >64 | >64 |
| enterica | | | | | | | | | | | |
| serovar | | | | | | | | | | | |
| Typhimurium | | | | | | | | | | | |
| CECT 443 | | | | | | | | | | | |
| Pseudomonas | 64 | 64 | 8 | >64 | >64 | >64 | >64 | >64 | >64 | >64 | >64 |
| aeruginosa | | | | | | | | | | | |
| ATCC | | | | | | | | | | | |
| 27853 | | | | | | | | | | | |
| P. aeruginosa | 64 | >64 | ND | >64 | >64 | >64 | >64 | >64 | >64 | >64 | >64 |
| 33b (PDR) | | | | | | | | | | | |
| Acinetobacter | 64 | 64 | ND | >64 | >64 | >64 | >64 | 64 | 64 | >64 | >64 |
| baumannii | | | | | | | | | | | |
| ATCC | | | | | | | | | | | |
| 19606 | | | | | | | | | | | |
| Campylobacter | 64 | 64 | ND | 64 | 64 | >64 | >64 | >64 | >64 | >64 | >64 |
| jejuni | | | | | | | | | | | |
| ATCC | | | | | | | | | | | |
| 33560 | | | | | | | | | | | |
| C. jejuni | 32 | 32 | 8 | >64 | >64 | >64 | >64 | >64 | >64 | >64 | >64 |
| P5/4 | | | | | | | | | | | |
| C. jejuni | 64 | 64 | ND | 64 | >64 | >64 | >64 | >64 | >64 | >64 | >64 |
| 4432 | | | | | | | | | | | |
| C. jejuni | 64 | 64 | ND | >64 | >64 | >64 | >64 | >64 | >64 | >64 | >64 |
| 4433 | | | | | | | | | | | |
| C. jejuni | 64 | 64 | ND | >64 | >64 | >64 | >64 | >64 | >64 | >64 | >64 |
| 4448 | | | | | | | | | | | |
| Yersinia | 16 | 32 | ND | >64 | >64 | >64 | >64 | 64 | 64 | >64 | >64 |
| ruckeri | | | | | | | | | | | |
| ATCC | | | | | | | | | | | |
| 29473 | | | | | | | | | | | |
| Listonella | 16 | 32 | ND | >64 | >64 | >64 | >64 | 32 | 32 | >64 | >64 |
| anguilarum | | | | | | | | | | | |
| ATCC 1924 | | | | | | | | | | | |
| Tenacibaculum | ND | ND | 20 | 18 | ND | 10 | ND | 10 | ND | 10 | ND |
| maritimum | | | | | | | | | | | |
| ATCC | | | | | | | | | | | |
| 43397* | | | | | | | | | | | |
| Staphylococcus | 16 | 16 | 13 | 32 | >64 | >64 | >64 | 64 | >64 | 16 | >64 |
| aureus | | | | | | | | | | | |
| ATCC | | | | | | | | | | | |
| 29213 | | | | | | | | | | | |
| S. aureus | 32 | 32 | 11 | 32 | >64 | >64 | >64 | 64 | >64 | 64 | >64 |
| 66/1 | | | | | | | | | | | |
| (MRSA) | | | | | | | | | | | |
| Enterococcuss | 16 | 32 | 13 | 32 | >64 | >64 | >64 | 64 | 64 | >64 | >64 |
| faecalis | | | | | | | | | | | |
| ATCC | | | | | | | | | | | |
| 29212 | | | | | | | | | | | |
| E. faecalis | 32 | 32 | 11 | >64 | >64 | >64 | >64 | 64 | >64 | >64 | >64 |
| B3/101 | | | | | | | | | | | |
| (VRE) | | | | | | | | | | | |
| Streptococcuss | 64 | 64 | ND | 32 | 32 | >64 | >64 | >64 | >64 | >64 | >64 |
| pyogenes | | | | | | | | | | | |
| ATCC | | | | | | | | | | | |
| 19615 | | | | | | | | | | | |

| | 6 | | 7 | | 8 | | 9 | |
|---|---|---|---|---|---|---|---|---|
| Strains | MIC | MBC | MIC | MBC | MIC | MBC | MIC | MBC |
| Escherichia | 64 | >64 | >64 | >64 | >64 | >64 | >64 | >64 |
| coli ATCC | | | | | | | | |
| 25922 | | | | | | | | |
| E. coli SA/2 | >64 | >64 | >64 | >64 | >64 | >64 | >64 | >64 |
| (ESBL) | | | | | | | | |
| E. coli 2252 | >64 | >64 | >64 | >64 | >64 | >64 | >64 | >64 |
| (COL-resistant) | | | | | | | | |
| Klebsiella | >64 | >64 | >64 | >64 | >64 | >64 | >64 | >64 |
| pneumoniae | | | | | | | | |
| ATCC 13883 | | | | | | | | |
| Salmonella | >64 | >64 | >64 | >64 | >64 | >64 | 64 | 64 |
| enterica | | | | | | | | |
| serovar | | | | | | | | |
| Typhimurium | | | | | | | | |
| CECT 443 | | | | | | | | |
| Pseudomonas | >64 | >64 | >64 | >64 | >64 | >64 | >64 | >64 |
| aeruginosa | | | | | | | | |
| ATCC | | | | | | | | |
| 27853 | | | | | | | | |
| P. aeruginosa | >64 | >64 | >64 | >64 | >64 | >64 | >64 | >64 |
| 33b (PDR) | | | | | | | | |
| Acinetobacter | 64 | 64 | >64 | >64 | >64 | >64 | 64 | 64 |
| baumannii | | | | | | | | |
| ATCC | | | | | | | | |
| 19606 | | | | | | | | |
| Campylobacter | >64 | >64 | >64 | >64 | >64 | >64 | >64 | >64 |
| jejuni | | | | | | | | |
| ATCC | | | | | | | | |
| 33560 | | | | | | | | |
| C. jejuni | >64 | >64 | >64 | >64 | >64 | >64 | >64 | >64 |
| P5/4 | | | | | | | | |
| C. jejuni | >64 | >64 | >64 | >64 | >64 | >64 | >64 | >64 |
| 4432 | | | | | | | | |
| C. jejuni | >64 | >64 | >64 | >64 | >64 | >64 | >64 | >64 |
| 4433 | | | | | | | | |
| C. jejuni | >64 | >64 | >64 | >64 | >64 | >64 | >64 | >64 |
| 4448 | | | | | | | | |
| Yersinia | >64 | >64 | >64 | >64 | >64 | >64 | >64 | >64 |
| ruckeri | | | | | | | | |
| ATCC | | | | | | | | |
| 29473 | | | | | | | | |
| Listonella | 64 | 64 | >64 | >64 | >64 | >64 | 64 | 64 |
| anguilarum | | | | | | | | |
| ATCC 1924 | | | | | | | | |
| Tenacibaculum | 10 | ND | 9 | ND | 12 | ND | 9 | ND |
| maritimum | | | | | | | | |
| ATCC | | | | | | | | |
| 43397* | | | | | | | | |
| Staphylococcus | 64 | 64 | 8 | >64 | 16 | >64 | 64 | 64 |
| aureus | | | | | | | | |
| ATCC | | | | | | | | |
| 29213 | | | | | | | | |
| S. aureus | 64 | 64 | 16 | >64 | >64 | >64 | 64 | 64 |
| 66/1 | | | | | | | | |
| (MRSA) | | | | | | | | |
| Enterococcuss | 64 | 64 | >64 | >64 | >64 | >64 | 64 | 64 |
| faecalis | | | | | | | | |
| ATCC | | | | | | | | |
| 29212 | | | | | | | | |
| E. faecalis | >64 | >64 | >64 | >64 | >64 | >64 | 32 | >64 |
| B3/101 | | | | | | | | |
| (VRE) | | | | | | | | |
| Streptococcuss | >64 | >64 | >64 | >64 | >64 | >64 | >64 | >64 |
| pyogenes | | | | | | | | |
| ATCC | | | | | | | | |
| 19615 | | | | | | | | |

COL, colistin
ESBL, extended-spectrum β-lactamase-producing strain
MIC, minimal inhibitory concentration
MBC, minimal bactericidal concentration
MRSA, methicillin-resistant Staphylococcus aureus
ND, not determined
PDR, pan-drug-resistant
VRE, vancomycin-resistant Enterococcus.
*The MIC of the compounds on T. maritimum was only determined by disk diffusion, as described in the CLSI guideline

Derivatives 1, 4, 6, and 9 exhibited activities against Gram-negative bacteria. Specifically, compounds 1, 4, 6, and 9 displayed an inhibitory effect against A. baumannii ATCC 19606. For Salmonella enterica serovar Typhimurium CECT 443, compounds 1 and 4 exhibited antimicrobial activity. For C. jejuni ATCC 33560, C. jejuni 4432, C. jejuni 4433 and C. jejuni 4448, compounds 1 and 2 showed antimicrobial activity. Compound 1 was also active against E. coli ATCC 25922, E. coli SA/2, E. coli 2252, K. pneumoniae ATCC 13883, P. aeruginosa ATCC 27853, P. aeruginosa 33b, and C. jejuni P5/4.

Concerning the Gram-positive strains, every compound except compound 3 showed activity. For S. aureus ATCC 29213, compounds 1, 2, 4-9 were active. In the case of S. aureus 66/1, compounds 1, 2, 4-7, and 9 showed antibacterial activity. Compounds 1, 2, 4, 6, and 9 exhibited antimicrobial activity for E. faecalis ATCC 29212, while 1, 4, and 9 displayed the same effect for E. faecalis B3/101. Lastly, compounds 1 and 2 was active against S. pyogenes ATCC 19615.

The compounds were also tested in Gram-negative fish pathogens. Compound 1 and 4 displayed antibacterial activity in Yersinia ruckeri ATCC 29473. In the case of Listonella anguilarum, compounds 1, 4, 6, and 9 were shown to be active. For Tenacibaculum maritimum ATCC 43397, all the tested compounds except 3 displayed growth inhibition.

The compounds displayed bactericidal and/or bacteriostatic activity, as illustrated by the MBC. When the MBC is greater than 64 μg/mL, the compound is bacteriostatic, otherwise it is bactericidal.

Compound 1 presents the broader spectrum, as it is active in every bacterial strain tested. It is also the only active compound in the E. coli, K. pneumoniae and P. aeruginosa strains tested. It was also the most effective compound in every strain tested, except for the S. aureus strains. Compound 7 displayed the lowest minimum inhibitory concentration for the S. aureus strains tested.

The activity of the compounds was, overall, greater for the reference strains, except for compound 9, which displayed a greater effect on the VAN-resistant E. faecalis than in the reference strain.

It was also shown that most compounds were active against Gram-positive strains, specifically S. aureus. A. baumannii was the Gram-negative bacteria towards which more compounds were active.

Regarding antibacterial activity, the structure-activity relationship (SAR) study suggested that the presence of a primary amine favors Gram-negative activity, as evidenced by compound 1. The presence of an electron withdrawing group hinders the antibacterial activity, as observed in the case of compound 3. It was also shown that the presence of a dimer can promote antibacterial activity, as compound 4 was active in eight of the tested strains. The presence of an amine at the terminal of the molecule can also be a favorable feature, as compound 9 also shows a broad spectrum of activity. And lastly, aliphatic substituents seem to be preferable over aromatic.

### Antibacterial Mechanism of Action

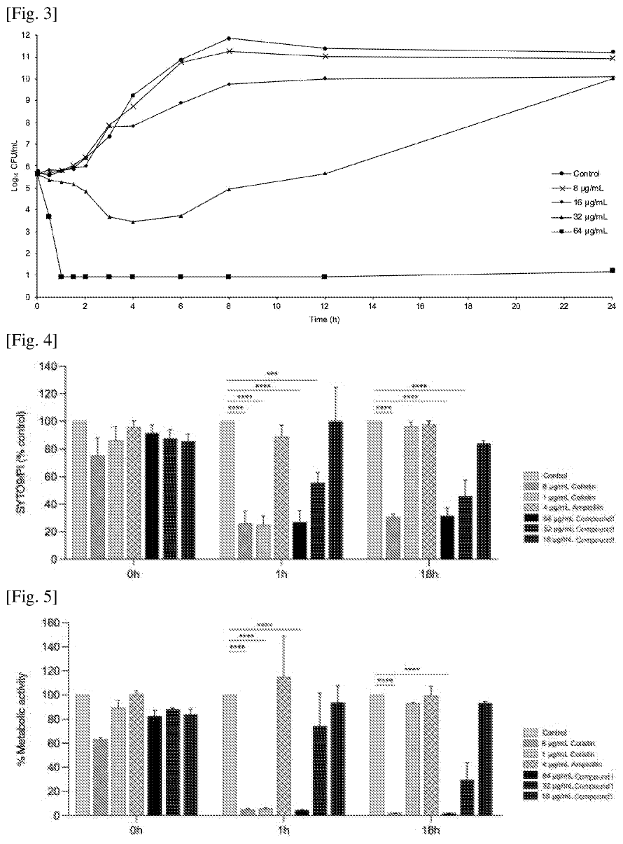

The present disclosure relates to antibacterial mechanism of action of the compound 1 herein disclosed. In an embodiment, the evaluation of time-kill kinetics was performed as follows. Time-kill kinetics of compound 1 were evaluated for E. coli ATCC 25922, allowing the confirmation of its bactericidal effect. This is usually determined when ≥99.9% killing of the initial inoculum occurs and is determined by a 3-log₁₀-unit decrease in CFU/mL. Bactericidal activity against E. coli ATCC 25922 was achieved after approximately 1 h of exposure to 64 μg/mL of compound 1 (2×MIC) ([FIG. 3]), however, after 30 min of exposure, a 2-log₁₀-unit decrease in CFU/mL was observed. From time-kill plots it is also possible to ascertain whether an antimicrobial agent produces concentration-dependent killing or time-dependent killing, for compound 1 it was possible to observe a concentration-dependent killing, as the extent of killing increases with higher drug concentrations. Time plays an important role for lower concentrations, as the compound seems to slow down the rate at which E. coli ATCC 25922 enters exponential phase at MIC (32 μg/mL) and ½×MIC (16 g/mL).

[FIG. 3] shows the determination of E. coli ATCC 25922 killing kinetics, which was carried out using the time-kill method, following CLSI guidelines. The effect of compound 1 concentrations ranging between 64 and 8 μg/mL was evaluated. The bacterial inoculum was prepared by suspending colonies from overnight cultures (grown in MHA) in CAMHB, and an aliquot of this suspension was added to each tube of CAMHB alone (control) or CAMHB plus an appropriate amount of compound 1, to give an inoculum of approximately 5×10⁵ CFU/mL, in a final volume of 10 mL. Tubes were incubated at 37 degrees C. in a water bath with shaking. At predetermined time points (30 min, 1, 1.5, 2, 3, 4, 6, 8, 12, and 24 h), 100 μL aliquots were aseptically removed from each tube, after vortexing, serially diluted in buffered peptone water (Biokar Diagnostics, Allone, Beauvais, France), if necessary, and spread on MHA. Colony counts were determined following overnight incubation at 37 degrees C. and log₁₀ CFU/mL was plotted against time. Three independent experiments were performed.

### Mode of Action

Evaluation of the Integrity of E. coli Cell Membrane Through the Propidium Iodide Influx Assay

Ceragenins have been described to interfere with the integrity of bacterial membranes [8,19], so this was investigated as a potential mode of action of compound 1. Loss of membrane integrity alters its permeability, which can be measured by propidium iodide (PI) influx, a fluorescent nucleic acid stain that can only penetrate damaged membranes [20].

The influx of PI in E. coli ATCC 25922 treated with 2×MIC, MIC, and ½×MIC of compound 1 was evaluated using a commercial kit that includes fluorescent nucleic acid stains SYTO® 9 and PI. SYTO® 9 can enter cells with intact or damaged membranes, generally labeling both, whereas PI only penetrates cells with severe membrane lesions, causing a reduction in SYTO® 9 fluorescence when both dyes are present.

In these assays, COL and AMP were used as controls. COL is a polycationic antimicrobial peptide with a complex mode of action, that ultimately solubilizes the bacterial cell membrane. AMP is an aminopenicillin that inhibits cell wall synthesis by inhibiting penicillin-binding proteins. E. coli ATCC 25922 cells were treated with compound 1 and, at several time-points (0, 1, and 18 h), samples were taken, and SYTO® 9/PI ratio was determined ([FIG. 4]). The entrance of PI is reflected in a reduction of this ratio. The time-points selected included the starting point (0 h); 1 h as at this time, for the bactericidal concentration (64 μg/mL, 2×MIC), there were no culturable cells, as determined in the time-kill assay; 18 h, as this was the incubation time for MIC determinations. The reduction of SYTO® 9/PI ratio, when compared to untreated control, was statistically significant for cells treated with 8 μg/mL of COL, but not for cells treated with 4 μg/mL AMP, which is consistent with the effect of these antibiotics, as described above. For 1 μg/mL of COL, there was only a significant reduction at 1 h, but not after 18 h of exposure, which is consistent with its effect on COL-sensitive strains.

The effect of exposure to compound 1 was dose-dependent and suggested a disruption in membrane integrity at 64 μg/mL (2×MIC, MBC), as the reduction in SYTO® 9/PI was significant at all time points, and similar to what was observed for a supra-inhibitory concentration of COL. After 1 and 18 h of exposure, the effect of compound 1 was also significant at 32 μg/mL (MIC).

[FIG. 4] shows the membrane integrity assessment through the PI influx assay, using a commercial kit (LIVE/DEAD® BacLight™ Bacterial Viability Kit, for microscopy & quantitative assays, Invitrogen by Thermo Fisher Scientific, Waltham, MA, USA), which includes fluorescent nucleic acid stains SYTO® 9 and PI. Briefly, E. coli ATCC 25922 colonies from overnight cultures in MHA were suspended in CAMHB to prepare an inoculum with a final concentration of approximately 5×10⁵ CFU/mL. The tubes were incubated overnight at 36° C. in a water bath with shaking. Following this, the cell suspensions were centrifuged at 3500 rpm for 15 min, the supernatant was removed, the cells were carefully resuspended in fresh CAMHB and the suspension was adjusted to 0.5 McFarland. An aliquot of this suspension was then added to each tube of CAMHB alone (control) or CAMHB plus an appropriate amount of test compound. Concentrations of compound 1 ranging between 64 and 16 μg/mL (2×MIC and ½×MIC) were tested. Positive controls included 8 μg/mL and 1 μg/mL COL (8×MIC and MIC) and 4 μg/mL AMP (MIC). The tubes were incubated at 36 degrees C. in a water bath with shaking and, at pre-established time points (0, 1, and 18 h), 500 L aliquots were retrieved for further analysis. After the exposure time, cells were washed two times, resuspended in 0.85% NaCl, and 100 μL of each cell suspension was distributed in the wells of a microtiter plate, in triplicate. 100 μL of a mixture of 30 μM PI and 5 μM SYTO® 9 prepared in ultrapure water were added to each well, and the plates were incubated at room temperature, in the dark, for 15 min. The fluorescence intensity of the stained bacterial suspensions was determined using a microplate reader (Synergy HT, BioTek Instruments, Winooski, VT, USA) by two consecutive measurements with excitation wavelength 485 nm and emission wavelength 528 nm (SYTO® 9) or 590 nm (PI). Data were analyzed by subtracting background fluorescence from each sample, dividing the fluorescence intensity of SYTO® 9 by fluorescence intensity of PI, and the results are presented as a percentage of control. Three independent assays were performed in triplicate for each experimental condition.

### Effect on Viability Assessed by Enzymatic Activity Measurements

To determine whether compound 1 could also affect enzymatic activity, 2-(4,5-dimethyl-2-thiazolyl)-3,5-diphenyl-2H-tetrazolium bromide (MTT) assay was performed. This assay can also be used as an indirect measure of cellular viability, as tetrazolium salts are reduced to purple formazan by metabolically active cells. E. coli ATCC 25922 cells were treated with compound 1 and COL and AMP were used as controls, as described above. Measurements were made at 0, 1, and 18 h. For controls treated with 8 μg/mL COL, there was a significant reduction of metabolic activity at all time points, while for cells exposed to 1 μg/mL COL there was only a significant reduction at 1 h, but not after 18 h of exposure. Treatment with 4 μg/mL AMP did not affect the metabolic activity of E. coli ATCC 25922.

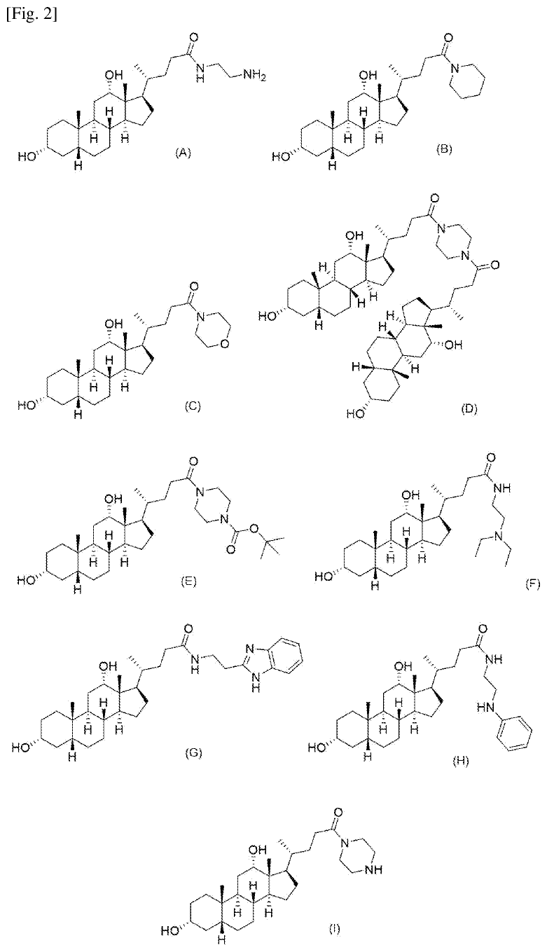

Metabolic activity of cells treated with 64 μg/mL compound 1 was significantly reduced at all time points, to similar levels of cells treated with 8 μg/mL COL. Lower concentrations of compound 1 did not affect E. coli ATCC 25922 in a significant manner, however, after 1 and 18 h of exposure to 32 μg/mL, there was a decrease in enzymatic activity. This assay is also an indirect measure of cellular viability and, as observed in time-kill assays ([FIG. 2]), after 1 h of exposure to 64 μg/mL of compound 1, there is a significant reduction of viability ([FIG. 5]).

### Effect on Viability Assessed by Enzymatic Activity Measurements

Enzymatic activity was evaluated by the MTT assay, as previously described, with some modifications. Cell suspensions of E. coli ATCC 25922 were prepared as described above, as well as test conditions and controls. At each time point (0, 1, and 18 h), 500 μL aliquots were collected, centrifuged at 3500 rpm for 10 min, the supernatant was removed, and 300 μL of MTT solution (0.5 mg/mL in CAMHB, at 36 degrees C.) (Thiazolyl Blue Tetrazolium Bromide, Alfa Aesar, Ward Hill, MA, USA) were added. After a 30 min incubation at 36° C., the insoluble formazan product resulting from the conversion of MTT by metabolically active cells was solubilized with 300 μL of DMSO. 100 μL of each sample were transferred in duplicate to the wells of a microtiter plate and the extent of the reduction of MTT to formazan was quantified by measuring the absorbance at 570 nm (Thermo Scientific Multiskan® EX, Thermo Fisher Scientific, Waltham, MA, USA). Three independent assays were performed in duplicate, and results are expressed as a percentage of untreated control.

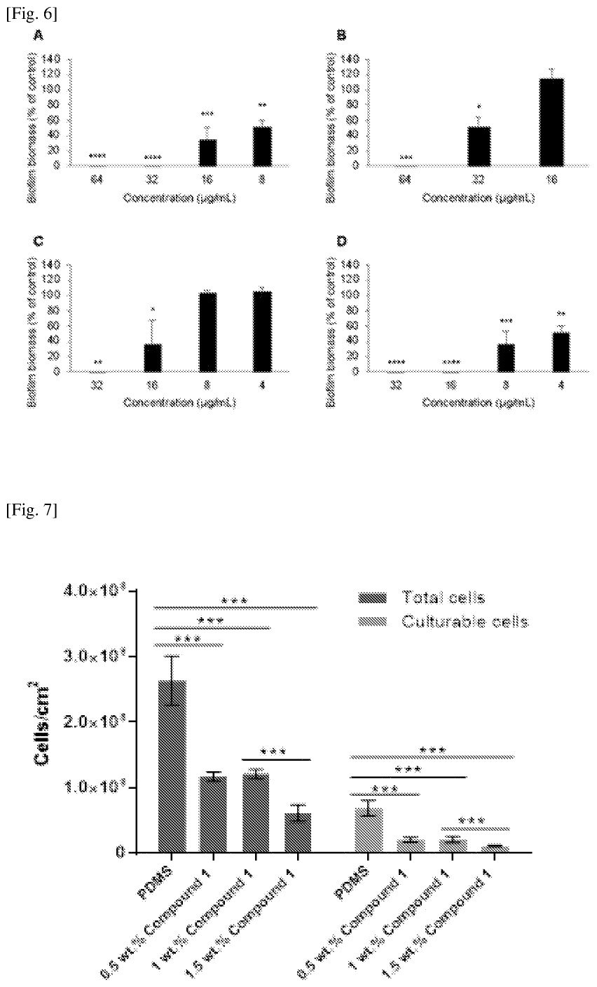

In an embodiment, the antibiofilm activity of compound 1 was performed as follows. Given its bactericidal effect, the impact of compound 1 on the biofilm formation of reference strains was studied. Supra- and sub-MIC concentrations were tested when possible, maintaining DMSO concentrations below 1%. Overall, bacterial biofilms were significantly inhibited at 2×MIC and MIC concentrations ([FIG. 6]). For E. coli ATCC 25922 and E. faecalis ATCC 29212, minimal biofilm inhibitory concentrations (MBIC), which is defined as the minimum compound concentration that leads to an 80% reduction of biofilm formation when compared to the untreated control, were below ¼×MIC (8 and 4 μg/mL, respectively).

### Antibiofilm Activity—Bacterial Biofilm Formation Inhibition Assay

The effect of compound 1 on bacterial biofilm formation was evaluated through quantification of total biomass, using the crystal violet method, as previously described ([FIG. 6]). Briefly, compound 1 in concentrations ranging between 2×MIC and ¼×MIC was added to bacterial suspensions of 1×10⁶ CFU/mL prepared in unsupplemented Tryptone Soy broth (TSB-Biokar Diagnostics, Allone, Beauvais, France) or TSB supplemented with 1% (p/v) glucose (D-(+)-Glucose anhydrous for molecular biology, PanReac AppliChem, Barcelona, Spain) for Gram-positive strains. A control with an appropriate concentration of DMSO, as well as a negative control (TSB alone) was included. Sterile 96-well flat-bottomed untreated polystyrene microtiter plates were used. After a 24 h incubation at 37 degrees C., the biofilms were heat-fixed for 1 h at 60 degrees C. and stained with 0.5% (v/v) crystal violet (Quimica Clinica Aplicada, Amposta, Spain) for 5 min. The stain was resolubilized with 33% (v/v) acetic acid (Acetic acid 100%, AppliChem, Darmstadt, Germany) and the biofilm biomass was quantified by measuring the absorbance of each sample at 570 nm in a microplate reader (Thermo Scientific Multiskan® EX, Thermo Fisher Scientific, Waltham, MA, USA). The background absorbance (TSB or TSB+1% glucose without inoculum) was subtracted from the absorbance of each sample and the data are presented as a percentage of control. Three independent assays were performed for reference strains, with triplicates for each experimental condition.

### Polydimethylsiloxane-Based Coating Containing Compound 1

Polydimethylsiloxane (PDMS) is one of the most used silicon-based organic polymers for the fabrication of medical implants, mainly due to its chemical stability, elastomeric and biocompatible properties, allied to its biomechanical behavior similar to biological tissues. In particular, PDMS-based coatings formulations (e.g. Sylgard™ 184) have been widely used for the development of new surfaces and functional protective coatings with potential application in urinary tract devices. To evaluate compound 1's potential as an antimicrobial additive for PDMS coating matrices and to contribute to the development of protective antimicrobial surfaces in a ureteral stent environment, a Sylgard™ 184 system was used to prepare coatings containing compound 1 at different contents (0.5, 1.0, and 1.5 wt %). However, due to incompatibility issues of compound 1 with the base resin component of the Sylgard™ 184 system formulation and further curing of the PDMS-based system, a pre-treatment surface step was performed on compound 1. For this purpose, the conventional GLYMO epoxy silane crosslinker was used. The nature of this bifunctional agent, also known as a silane treatment agent of general formula R(4-x)Si(OR′)x, wherein x is an integer of 1 to 3; OR′ is a hydrolyzable group such as methoxy, R is an organic functional group such as an epoxy group, allows it to chemically bond dissimilar materials. The epoxy groups of its structure interact with amino groups, while the methoxy silane groups interact with the resin-based matrix. This silane treatment has been widely used for this purpose, and since earlier showed the ability to covalent attach small-molecule antimicrobials via an alkoxysilane tethering. The silane treated compound 1 with GLYMO was further added and blended, as a dispersion, in the coating Sylgard™ 184 system. The obtained formulations were used to coat glass inserts (1×1 cm) to perform dynamic biofilm assays.

### Preparation of the Polydimethylsiloxane (PDMS)-Based Coatings Containing Compound 1

Compound 1 immobilization in PDMS-based coatings followed a two steps procedure. The first step comprises the pre-treatment surface of compound 1 with the GLYMO epoxy silane crosslinker, where to a 0.05 M solution of compound 1 in Me-THF (99%, Alfa Aesar) was added the GLYMO (≥98%, Sigma Aldrich) in a GLYMO/compound 1 molar ratio of 1.5. The resulted mixture was heated and maintained overnight at 40° C. under an inert atmosphere and continuous stirring. After this period the reaction was stopped, and the solvent was removed in a Butchi R210/215 rotavapor. The obtained precipitated was washed with Me-THF and dried in an oven at 40 degrees C. to originate the compound 1-M. Anal. Calcd for C₃₅H₆₆N₂O₈Si: C, 62.65; H, 9.91; N, 4.17; O, 19.07; Si, 4.19. Found: C, 50.20; H, 8.47; N, 2.42; Si, 9.78. The second step comprises the direct incorporation of the obtained compound 1 M in a Sylgard™ 184 system. For this purpose, compound 1-M was prior dispersed (ca 4.0 wt %) in ethyl(−)-L-lactate EMPLURA® (p.a, Sigma-Aldrich) promoted by an ultrasonic bath, further added and blended in the base resin and curing agent components (base/curing agent ratio=10/1) of the Sylgard™ 184 coating formulation system. The incorporated amount of compound 1-M dispersion into the PDMS-based system respected the desirable content in the wet coating formulation, 0.5, 1.0, and 1.5 wt %. The final obtained Sylgard™ 184 formulations were further used to coat 1×1 cm glass substrates through a dipping coating methodology. Thirty substrates were coated for each prepared formulation, including the pristine PDMS control coating formulation for the dynamic bacterial biofilm formation assays.

E. coli Biofilm Formation Under Hydrodynamic Conditions

It is known that bacterial adhesion and biofilm formation are influenced by several biological factors, including physiological conditions, pH, and shear stress. To mimic the conditions found in ureteral stents, biofilm experiments were performed using an E. coli ATCC 25922 suspension of approximately 7.6×10⁷ cells/mL prepared in synthetic urine and incubated with PDMS (control) and compound 1-M films at 37 degrees C. at the critical shear stress range for incrustation in ureteral stents (0.01-0.02 Pa).

After 48 h of biofilm growth, the total number of sessile cells on samples was determined by staining the biofilm suspension with 4′-6-diamidino-2-phenylindole (DAPI) and analyzing in a fluorescence microscope, while the cell culturability was assessed by spreading the biofilm suspension on an agar plate followed by colony-forming unit counting. Results obtained for total and culturable cell quantification are shown in [FIG. 7]. There were statistically significant differences in the number of total and culturable cells between the PDMS control and compound 1-M based PDMS films (p<0.001). Biofilms formed on PDMS films had, on average, 2.64×10⁸ cell/cm², and 26% of these cells were culturable. The incorporation of 0.5, 1.0, and 1.5 wt % compound 1 on PDMS films resulted in a decrease of E. coli biofilm formation of 55.6, 54.4, and 76.9%, respectively. Likewise, the viability of E. coli biofilm cells growing on compound 1-M based PDMS films was reduced by 70.1-83.5% compared with control. Significant differences for the number of total and culturable cells between 0.5 and 1.0 wt % compound 1 were not observed (p=0.275 and 0.965, respectively). However, 1.5 wt % compound 1-M based PDMS films reduced the number of total and culturable cells by approximately 23 and 14% (p<0.001), respectively, in comparison with 1.0 wt % compound 1-M based PDMS films. These results indicate that the incorporation of compound 1 into PDMS coating matrix, one of the most widely used materials for the constructions of urinary tract devices, significantly reduced the E. coli biofilm formation and that the antibiofilm efficacy of compound 1-based PDMS M films depends on the compound 1 content.

### Dynamic Bacterial Biofilm Formation Assay

A cryo-preserved batch of E. coli ATCC 25922 (stored in glycerol at −80 degrees C.) was firstly cultured on plate count agar (PCA, Merck, Germany) at 37 degrees C. Afterward, an overnight culture of E. coli ATCC 25922 was prepared by adding few colonies of this previously prepared culture to 125 mL of artificial urine medium (AUM) and further incubation at 37 degrees C. and 120 rpm. Cell density was then adjusted to an optical density (OD) of ˜0.1 at 610 nm (equivalent to a bacterial concentration of approximately 7.6×10⁷ CFU/mL). After that, a total of 3 mL of cell suspension was transferred into each well of a sterile 12-well flat-bottomed untreated polystyrene microtiter plate containing the films produced as described in the previous section (all the surfaces were previously sterilized by UV light for 30 min). Biofilm formation was allowed to occur by incubating the microtiter plates (including a negative control composed by AUM medium) at 37° C. and 100 rpm. The maximum shear stress at the surface of the films was 0.019 Pa, as determined by computational fluid dynamics. Given that E. coli biofilms in urinary devices are mature after 24 h, after 48 h of biofilm growth, the cell suspension was removed, and the films were carefully washed with 3 mL of sterile saline solution (8.5 g/L NaCl) to eliminate the remaining non-adherent cells. The films were then promptly transferred to 2 mL of sterile saline and vigorously vortexed for 3 min to promote the mechanical detachment of the biofilm from the upper face of the film. The total cell number was determined by staining suspended biofilm cells with 4′-6-diamidino-2-phenylindole (DAPI, Merck, Germany), which stains both viable and non-viable cells [62], followed by the observation of stained cells with the aid of an epifluorescence microscope (Leica DM LB2) connected to a camera (Leica Microsystems Ltd., Switzerland). Total cell counts were predicted from the analysis of a minimum of 15 fields of view and the final values are presented as total cells/cm². To assess cell viability, serial decimal dilutions of the biofilm suspensions were prepared, plated on PCA, and incubated at 37 degrees C. for colony enumeration. Biofilm cell counts are reported as CFU per unit of surface area (CFU/cm²). Two independent experiments were performed for each surface, with a triplicate set of coupons or glass inserts for each experimental condition ([FIG. 7]).

### Antifungal Activity

The antifungal activity of compound 1 was evaluated for a wide range of fungal species, covering yeasts and filamentous fungi, including dermatophytes. Compound 1 was shown to have a broad spectrum of activity, as it was active against all microorganisms tested (Table 2), including sensitive strains and MDR strains. This antimicrobial effect was fungicidal, with minimal lethal concentrations (MLC) being equal to, or one or two-fold higher than the respective MIC. Twenty-three fungal strains were used in this study, including reference strains and clinical isolates of yeasts and filamentous fungi. Yeast strains included reference strains (ATCC—American Type Culture Collection and CECT—Colección Española de Cultivos Tipo) and clinical isolates: C. albicans ATCC 10231, Candida krusei ATCC 6258, C. albicans H37, C. albicans FF172, C. albicans FF176, C. albicans DSY294, C. albicans DSY296, C. glabrata DSY562, C. glabrata DSY565 and Cryptococcus neoformans CECT 1078. Filamentous fungi included Aspergillus fumigatus ATCC 240305, A. fumigatus C111, A. niger ATCC 16404, A. flavus F44, Fusarium solani FF125, F. oxysporum FF115, and dermatophytes Trichophyton rubrum FF5, T. mentagrophytes FF7, Microscoporum canis FF1 and Nannizzia gypsea FF3 (formerly Microsporum gypseum) and a species of genera Mucor, Lichetheimia and Scedosporium. Saprolegnia parasitica CBS 223.65, a reference strain of an oomycete fish pathogen, was also included. All microorganisms were kept in Sabouraud Dextrose Broth (SDB: BioMérieux, Marcy L'Etoile, France) plus glycerol (20%) at −80° C. The strains were kept in Sabouraud Dextrose Agar (SDA: BioMérieux, Marcy L'Etoile, France) slants and were sub-cultured in SDA before each test. S. parasitica was kept in Corn Meal Agar plates (CMA: BBL™ Corn Meal Agar, BD, Le Pont-de-Claix, France) and was sub-cultured in CMA before each experiment. C. albicans H37, was kindly provided by Cidilia Pina Vaz (CHSJ, Porto, Portugal) and C. albicans DSY294, C. albicans DSY296, C. glabrata DSY562, C. glabrata DSY565 were kindly provided by D. Sanglard (University of Lausanne, Switzerland). A stock solution of compound 1 (10 mg/mL), was prepared in dimethyl sulfoxide (DMSO 99%: Alfa Aesar, Kandel, Germany), kept at −20 degrees C., and freshly diluted in the appropriate culture media before each assay. In all experiments, in-test concentrations of DMSO were kept below 2%. Fluconazole (Alfa Aesar, Ward Hill, MA, USA) was tested as commercial antifungal.

Antifungal activity was evaluated by determining the MIC of compound 1 by the broth microdilution method, according to CLSI guidelines (reference documents M27-A3 for yeasts and M38-A2 for filamentous fungi). Briefly, cell or spore suspensions were prepared in RPMI-1640 broth medium (Biochrom, Berlin, Germany) buffered with 3-(N-morpholino)propane sulfonic acid (MOPS) (Sigma-Aldrich, St. Louis, MO, USA) (henceforth referred to as RPMI) from fresh cultures of the different strains of fungi. For yeasts, the inoculum was adjusted to 0.5-2.5×10³ CFU/mL. For filamentous fungi, the inoculum was adjusted to 1-3×10³ CFU/mL for dermatophytes, and 0.4-5×10⁴ CFU/mL for all other strains. Two-fold serial dilutions of the compound were prepared in RPMI, within the concentration range of 8-128 μg/mL. Sterility and growth controls were included in each assay. The 96-well flat-bottomed untreated polystyrene microtiter plates which were incubated for 48 h at 35° C., with the exception of Lichetheimia spp. which was incubated at 25° C. for 48 h, and the dermatophyte strains, which incubated for 5-7 days at 25° C. MICs were recorded as the lowest concentrations that completely inhibited growth in comparison to the compound-free controls. Voriconazole (kindly provided by Pfizer Ldt., UK) MIC for C. krusei ATCC 6258 was used as quality control and the assays were validated when the results obtained were within the recommended limits. The minimal lethal concentration (MLC) was determined by spreading 20 μL of culture collected from wells showing no visible growth on SDA plates. The MLC was determined as the lowest concentration showing complete growth inhibition after 48 h at 35 degrees C., 48 h at 25 degrees C. (Lichetheimia spp.) or 5-7 days at 25 degrees C. (dermatophytes). At least two independent assays were performed for all tested strains.

Antifungal activity of compound 1 against S. parasitica CBS 223.65 was evaluated by determining the MIC by a broth microdilution method carried out in Glucose Yeast (GY) Broth (10 g/L D-(+)-Glucose [Merck, Darmstadt, Germany]; 2 g/L Yeast extract [Liofilchem, Roseto Degli Abruzzi, Italy]). Briefly, two-fold serial dilutions of the compound were prepared in GY broth, within the concentration range of 8-128 μg/mL and 200 μL of each concentration were distributed in the wells of 96-well flat-bottomed untreated polystyrene microtiter plates. Each well was then inoculated with approximately 1 mm² of mycelium. Sterility and growth controls were included in each assay. After 5-7 days of incubation at 24° C., MIC was recorded as the lowest concentration that prevented obvious mycelial growth in comparison to the compound-free controls. Subsequently, the portions of the inocula in which growth was not observed were transferred to GY agar (GY broth with 15 g/L of agar [Agar Bios Special LL, Biolife, Milano, Italy]), after being washed two times in saline. The plates were incubated 5-7 days at 24 degrees C., in order to evaluate fungal viability and determine the MLC. At least four independent assays were performed.

TABLE 2
Antifungal activity of compound 1 against reference and clinical
fungal strains. MIC and MLC are expressed in μg/mL.

| | Compound 1 | | Fluconazole | |
|---|---|---|---|---|
| | (μg/mL) | | (μg/mL) | |
| | MIC | MLC | MIC | MLC |
| Candida albicans ATCC 10231 | 64 | 64 | 2 | >128 |
| C. albicans H37^{a} | 64 | 64 | ≥128 | >128 |
| C. albicans FF172 | 64 | 64 | 0.25 | 128 |
| C. albicans FF176^{a} | 32 | 32 | 32 | >128 |
| C. albicans DSY294 | 64 | 64 | 0.125 | 128 |
| C. albicans DSY296^{a} | 64 | 64 | 64 | >128 |
| C. krusei ATCC 6258^{b} | 64 | 64 | 32 | >128 |
| C. glabrata DSY562 | 64 | 64 | 4 | >128 |
| C. glabrata DSY565^{a} | 64 | 64 | 128 | >128 |
| Cryptococcus neoformans CECT | 32 | 64 | 8 | >32 |
| 1078 | | | | |
| Aspergillus fumigatus ATCC | 128 | ≥128 | ≥128 | >128 |
| 204305^{b} | | | | |
| A. fumigatus C111^{a,b} | 128 | >128 | ≥128 | >128 |
| A. niger ATCC 16404^{b} | 64 | 128 | ≥128 | >128 |
| A. flavus F44^{b} | ≥128 | >128 | 128 | >128 |
| Fusarium solani FF125^{b} | 64 | >128 | ≥128 | >128 |
| Fusarium oxysporum FF115^{b} | 128 | ≥128 | 64 | >128 |
| Mucor spp.^{b} | 64 | 64 | >128 | >128 |
| Lichtheimia spp.^{b} | 64 | >128 | 64 | >128 |
| Scedosporium spp. | 64 | 128 | 4 | 16 |
| Trichophyton rubrum FF5 | 32 | 64 | 16 | 64 |
| T. mentagrophytes FF7 | 32 | 64 | 8 | 32 |
| Microsporum canis FF1 | 64 | 128 | 32 | ≥128 |
| Nannizzia gypsea FF3 | 32 | 32 | 32 | ≥128 |
| Saprolegnia parasitica CBS 223.65 | 32-64 | 32-64 | — | — |

MIC, minimal inhibitory concentration
MLC, minimal lethal concentration
^{a}resistant to fluconazole or azoles
^{b}intrinsically resistant to fluconazole

C. albicans is the most frequent uropathogen fungi, with resistance to azoles being of rising concern, given the fact that these are the agents normally used to treat UTIs. Candida non-albicans species such as C. krusei and C. glabrata are also important due to their intrinsic resistance or reduced susceptibility to several antifungals, particularly to fluconazole. Urinary tract candidiasis is a very frequent nosocomial fungal infection, which usually occurs in patients with catheters and stents, typically after antibiotic therapy. As such, in Table 2 fluconazole MICs are also presented, illustrating that compound 1 has fungicidal activity against fungal strains with a wide range of MICs to this azole.

### Time-Kill Kinetics Evaluation

As mentioned for antibacterial activity, time-kill plots allow the evaluation of killing of a microbial isolate over time and establishing how much exposure time is needed in order to achieve a fungicidal effect, which is usually defined by ≥99.9% killing of the initial inoculum and is determined by a 3-log₁₀-unit decrease in CFU/mL. These curves are also used when evaluating whether a new antimicrobial agent produces concentration-dependent killing or time-dependent killing. Time-kill kinetics of compound 1 were evaluated for C. albicans ATCC 10231.

Determination of killing of C. albicans ATCC 10231 over time was carried out using the time-kill method, as previously described. This assay was performed for concentrations of compound 1 ranging between 64 and 8 μg/mL. Colonies from 24 h cultures in SDA were suspended in sterile saline and adjusted to 0.5 McFarland. An aliquot of this suspension was then added to each tube of RPMI alone (control) or RPMI plus an appropriate amount of compound 1, to give an inoculum of approximately 10⁵ CFU/mL in a final volume of 10 mL. Tubes were incubated at 36° C. in a water bath with shaking and vortexed prior to removing each sample for the determination of colony counts. At predetermined time points (30 min, 1, 1.5, 2, 3, 4, 6, 8, and 12 h), 100 μL aliquots were aseptically removed from each tube, serially diluted in sterile saline, and spread on SDA plates. Colony counts were determined following incubation at 36° C. for 24 to 48 h and log₁₀ CFU/mL was plotted against time. At least three independent experiments were performed.

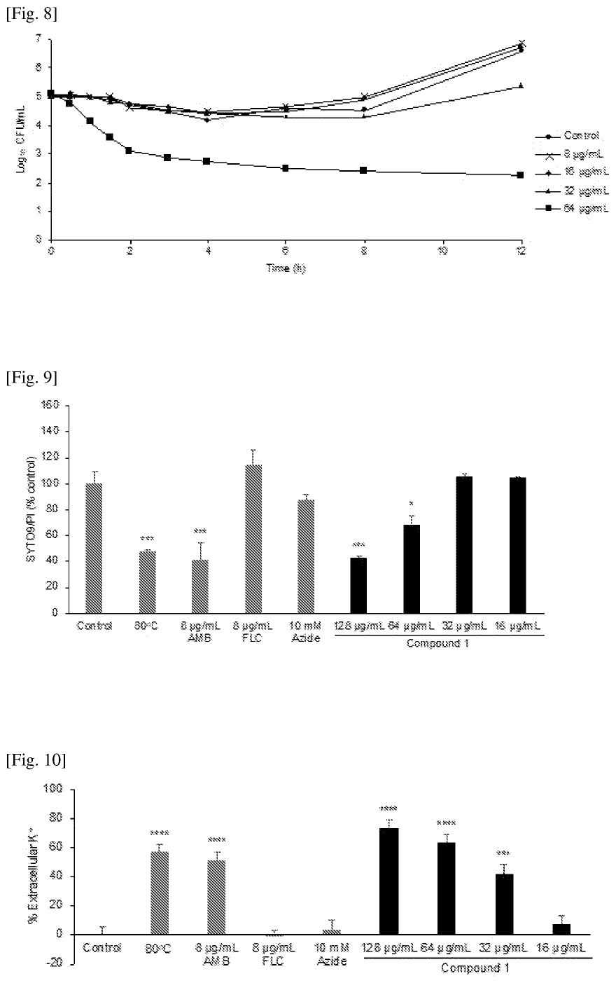

It was possible to observe a concentration-dependent killing for this compound, as the extent of killing increases with increased drug concentrations. Exposure to 64 μg/mL of compound 1 (MIC) results in a fungicidal effect against C. albicans ATCC 10231 after approximately 4 h, as evidenced in [FIG. 8]. Even though compound 1 has a concentration-dependent effect, its fungicidal concentration has a time-dependent effect, as until 2 h of exposure it can be observed a 1-log₁₀-unit decrease in CFU/mL each hour, which then slows down, taking an additional 10 h to produce an additional 1-log₁₀-unit decrease in CFU/mL counts.

### Mode of Action

As ceragenins have been described to disrupt fungal membranes, this was investigated as a potential mode of action of compound 1.

Evaluation of the Integrity of C. albicans Cytoplasmic Membrane

Membranes play a vital role in maintaining cellular structure and homeostasis, and compounds that compromise its integrity can lead to pore formation, leakage of intracellular content, and cell death. As such, compound 1 potential mode of action was primarily evaluated by measuring PI influx, a fluorescent nucleic acid stain that only penetrates damaged membranes, and by measuring the efflux of intracellular potassium ions.

In these assays, several controls were used. Amphotericin B (AMB), a polyene with fungicidal activity, which binds to plasma membrane ergosterol, perforating it, leading to leakage of cytosol and cell death. Fluconazole (FLC), an azole with fungistatic activity, that inhibits ergosterol biosynthesis by interfering with the cytochrome P450-dependent enzyme lanosterol 14-alpha-demethylase, involved in the transformation of lanosterol into ergosterol, which leads to alterations in cell membrane structure, and inhibition of fungal growth and P450-dependent enzymes involved in fungal respiration. Sodium azide which kills yeast cells by interfering with their metabolic activity, but without affecting the integrity of the plasma membrane. In addition to being chemically disrupted, the yeast cells were also physically disrupted by incubation at 80 degrees C. for 20 min.

### Propidium Iodide Influx Assay

Influx of PI in C. albicans ATCC 10231 treated with compound 1 was evaluated using a commercial kit, which includes fluorescent nucleic acid stains SYTO™ 9 and PI, and measurements were conducted in a fluorescence microplate reader. As described for antibacterial activity, SYTO™ 9 and PI differ in their spectral characteristics as well as their ability to penetrate cell membranes: SYTO™ 9 generally labels microorganisms with intact membranes and those with damaged membranes, whereas PI only penetrates cells with severe membrane lesions, causing a reduction in SYTO™ 9 fluorescence when both dyes are present. The ability of PI to penetrate cells with damaged membranes makes it suitable for studying the effect of drugs on cell membranes.

In this assay, cells were treated with compound 1 for 5 min, before the SYTO® 9/PI ratio was determined ([FIG. 9]). Entrance of PI is reflected in a reduction of this ratio and was statistically significant for controls treated with AMB and with heat, but not FLC and sodium azide, when compared to an untreated control, which is consistent with the effect of these treatments in fungal cells, as described above. Regarding exposure to compound 1, the reduction of SYTO® 9/PI ratio was dose-dependent, and significant at 128 and 64 μg/mL (2×MIC and MIC, respectively), suggesting a disruption in membrane integrity at these concentrations. At 2×MIC, SYTO® 9/PI ratio is similar to controls treated with AMB and with heat.

Additionally, even though it takes 4 h to achieve a fungicidal effect, with ≥99.9% killing of the initial inoculum ([FIG. 9]), induction of membrane disruption is evidenced after only 5 min of exposure to compound 1 (FIGS. 8 and 9).

### Potassium Ion Efflux Analysis

Leakage of potassium ions is a common response to membrane-disrupting agents, therefore, extracellular K⁺ was quantified by flame atomic absorption spectrometry, after 5 min of exposure to compound 1. In order to assess membrane integrity by the PI influx assay, a commercial kit was used (LIVE/DEAD® BacLight™ Bacterial Viability Kit, for microscopy & quantitative assays, Invitrogen by Thermo Fisher Scientific, Waltham, MA, USA), which includes fluorescent nucleic acid stains SYTO® 9 and PI. Briefly, C. albicans ATCC 10231 colonies from overnight cultures in SDA were suspended in sterile saline and adjusted to 0.5 McFarland. This suspension was then diluted in RPMI (1:50 followed by 1:20) to achieve a final concentration of 0.5-2.5×10³ CFU/mL and the tubes were incubated overnight at 36° C. in a water bath with shaking. The following day, the cell suspensions were centrifuged at 3500 rpm for 15 min, the supernatant was removed, and the cells were carefully resuspended in 2 mL of 0.85% NaCl (VWR International, Radnor, PA, USA) prepared in ultrapure water. An aliquot of this suspension was then added to each tube of 0.85% NaCl alone (control) or 0.85% NaCl plus an appropriate amount of test compound, in a 1:10 proportion. Concentrations of compound 1 ranging between 128 and 16 μg/mL (2×MIC and ¼×MIC), were tested. Positive controls included 8 μg/mL amphotericin B (Sigma-Aldrich, St. Louis, MO, USA) and an additional positive control was prepared by incubating a sample prepared in 0.85% NaCl alone at 80° C. for 20 min. Negative controls included 8 μg/mL fluconazole and 10 mM sodium azide (Merck, Darmstadt, Germany). The tubes were incubated for 5 min at 36° C. in a water bath with shaking. After the exposure time, cells were centrifuged for 10 min at 3500 rpm, the supernatant was removed, and the cells were resuspended in 0.85% NaCl. Following an additional washing step, 100 μL of each cell suspension was distributed in the wells of a microtiter plate, in triplicate. To each well was then added 100 μL of a mixture of 30 μM PI and 5 μM SYTO® 9 prepared in ultrapure water, and the plates were incubated at room temperature in the dark for 15 min. Fluorescence intensity of the stained yeast suspensions was obtained in a microplate reader (Synergy HT, BioTek Instruments) by two consecutive measurements: with excitation wavelength 485 nm and emission wavelength 528 nm (SYTO® 9) and with excitation wavelength 485 nm and emission wavelength 590 nm (PI). Data were analyzed by subtracting background fluorescence from each sample, dividing fluorescence intensity of SYTO® 9 by fluorescence intensity of PI, and are presented as a percentage of control. Three independent assays were performed in triplicate for each experimental condition.

In an embodiment, the potassium ion (K⁺) efflux analysis was performed as follows ([FIG. 9]). Leakage of potassium ions is a common response to membrane-disrupting agents; therefore, extracellular K⁺ was quantified by flame atomic absorption spectrometry, after 5 min of exposure to compound 1 ([FIG. 10]). When compared to an untreated control, levels of extracellular K⁺ were statistically significant for heat-treated cells and AMB-treated cells, but not FLC and sodium azide-treated cells, which is consistent with the effect of these treatments in fungal cells. Regarding cells exposed to compound 1, there were found significant levels of extracellular K⁺ for 128, 64, and 32 μg/mL (2×MIC, MIC, and ½×MIC, respectively). Efflux of K⁺ was dose-dependent, and similar to what was determined for heat-treated cells and AMB-treated cells. Of note, the effect obtained for ½×MIC of compound 1 was similar to that obtained for 4×MIC of AMB. This extensive leakage of K⁺ supports the hypothesis that compound 1 causes membrane disruption. Interestingly, at ½×MIC there is no evidence of influx of PI ([FIG. 9]), but the efflux of K⁺ is significant ([FIG. 10]), which suggests the formation of smaller pores in the plasma membrane after 5 min of exposure to 32 μg/mL of compound 1. Efflux of potassium was determined by quantification of extracellular K⁺ by flame atomic absorption spectrometry. Cell suspensions of C. albicans ATCC 10231 were prepared in the same manner as described for PI influx assay, and test conditions were also the same. After 5 min of exposure, cells were centrifuged for 10 min at 3500 rpm and the supernatants were filtrated using a cellulose acetate syringe filter with a 0.22 m pore size. Samples were analyzed with a AAnalyst 200 Atomic Absorption Spectrometer (Perkin Elmer). Four independent assays were performed and the data are presented as percentage of control (untreated cells as 0% of K⁺).

### Ergosterol Binding Assay

Ceragenins have been described to interact with the lipophilic environment of microbial membranes and ergosterol is the major sterol component of fungal plasma membrane, and the target of several antifungals. As such, in order to evaluate the ability of compound 1 to bind to membrane ergosterol of C. albicans ATCC 10231, MICs were determined in the absence and presence of exogenous ergosterol ([FIG. 10]). If the tested compound has the ability to perturb membrane integrity by binding to ergosterol, is expected that, in the presence of exogenous ergosterol, the compound binds to it, decreasing the amount of compound available to bind to membrane ergosterol, therefore increasing the MIC. Amphotericin B was used as a positive control for this assay and MICs obtained were 8 times higher in the presence of 400 μg/mL of exogenous ergosterol than in its absence (2 to 16 μg/mL, data not shown). For compound 1, MICs were unaffected by the presence of exogenous ergosterol, suggesting that ergosterol is not the target of compound 1. However, this result is not unexpected, as compound 1 showed antibacterial activity in the same range of concentrations as its antifungal activity, and ergosterol is not present in their membranes. The ability of compound 1 to bind to membrane ergosterol of C. albicans ATCC 10231, was evaluated by the ergosterol binding assay, as previously described. Briefly, MICs of compound 1 were determined by broth microdilution, as described above, in the absence and presence of ergosterol (Sigma-Aldrich, St. Louis, MO, USA) at a final concentration of 400 μg/mL. Ergosterol was prepared immediately before being added to the plates, by maceration and dissolution in DMSO. The formed emulsion was then homogenized, heated to increase solubility, and diluted in RPMI. Amphotericin B was used as positive control. Plates were incubated at 35° C. for 48 h and MICs were determined as described above. At least two independent assays were performed.

### Effect on Mitochondrial Dehydrogenases Activity

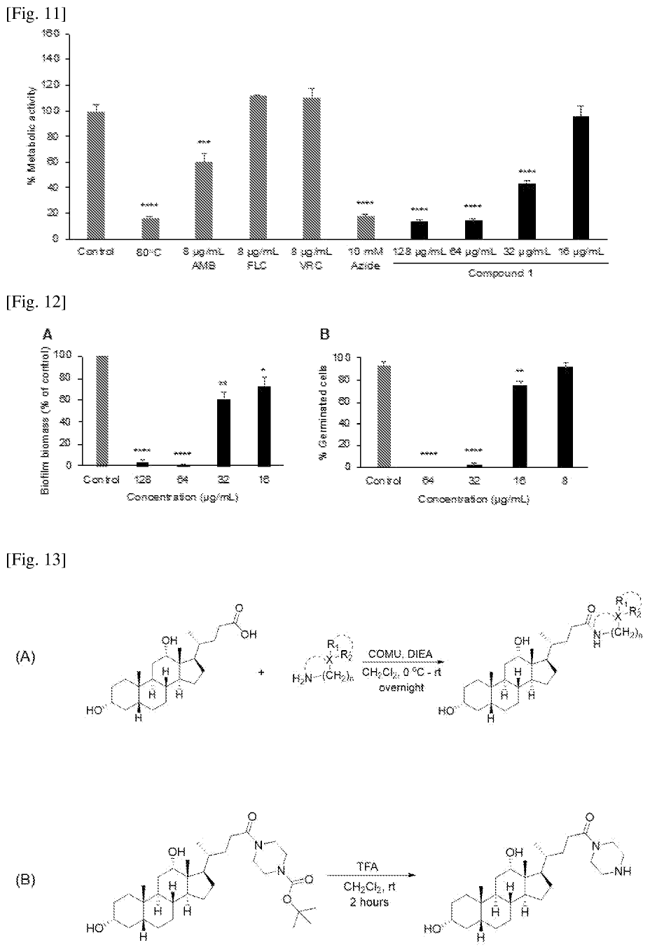

In order to determine whether compound 1 could also affect mitochondrial function, MTT reduction assay was performed. In this assay, tetrazolium salts are reduced to purple formazan derivatives by mitochondrial dehydrogenases, which can be measured spectrophotometrically and reported to mitochondrial activity and, indirectly, to cell viability. In this assay, cells were exposed to the test compounds for 2 h ([FIG. 11]). For the controls, cells treated with heat and with AMB and sodium azide, mitochondrial activity was significantly reduced. For FLC there was an increase (non-significant) in mitochondrial activity; voriconazole (VRC) was then added as a control, with the purpose of clarifying if this was common to other azoles, and an identical effect was observed. An increase in reactive oxygen species (ROS) has been linked to azoles activity: even though their primary mode of action is the inhibition of ergosterol synthesis, they also induce accumulation of ROS, which enhances their antifungal activity and translates into an increase in mitochondrial activity at inhibitory concentrations. For cells treated with compound 1 for 2 h, there was a significant reduction of mitochondrial activity found for cells treated with 128, 64, and 32 μg/mL (2×MIC, MIC, and ½×MIC). This inhibitory effect of mitochondrial function appears to be secondary to membrane disruption. Mitochondrial dehydrogenase activity was evaluated by the MTT assay, as previously described, with some modifications. Cell suspensions of C. albicans ATCC 10231 were prepared in the same manner as described above and, after overnight incubation at 36° C., were centrifuged at 3500 rpm for 15 min, the supernatant was removed, and the cells were carefully resuspended in 2 mL of RPMI. Test conditions and controls were the same as described above, but the time of exposure to the test compounds was 2 h. After the exposure time, cell suspensions were centrifuged at 3500 rpm for 10 min, the supernatant was removed, and 500 μL of MTT solution (0.5 mg/mL in RPMI, at 35° C.) (Thiazolyl Blue Tetrazolium Bromide, Alfa Aesar, Ward Hill, MA, USA) were added. After a 1 h incubation at 35° C., the insoluble formazan product resulting from the conversion of MTT by mitochondrial dehydrogenases of metabolically active cells was solubilized with 300 μL of DMSO. 100 μL of each sample were transferred in duplicate to a microtiter plate and the extent of the reduction of MTT to formazan was quantified by measuring the absorbance at 570 nm. Three independent assays were performed in duplicate, and the results are expressed as percentage of MTT reduction, using the untreated cells as control.

Synergy with Other Antifungal Drugs

The combined effect of compound 1 and amphotericin B and fluconazole for C. albicans ATCC 10231 were evaluated by the checkerboard method ([FIG. 11]). Fractional inhibitory concentration indices (FICI)≤0.5 were obtained for both antifungals. According to the consensus interpretation of FICI values, FICI≤0.5 is interpreted as ‘synergy’. Albeit FICI obtained were in the higher frontier of synergy, is relevant to note that 16 μg/mL of compound 1 (¼×MIC) eliminated trailing caused by fluconazole at its MIC (2 μg/mL). Whereas for AMB there appears to be an association of effects between the antifungal and compound 1. The synergy between compound 1 and amphotericin B or fluconazole was evaluated for C. albicans ATCC 10231, by the checkerboard method, as previously described. Inoculum and compound solutions were prepared as described for antifungal susceptibility testing. Fractional inhibitory concentrations (FIC) were calculated as follows: FIC of compound=MIC of compound combined with antifungal/MIC compound alone, and FIC antifungal=MIC of antifungal combined with compound/MIC of antifungal alone. The FIC index (FICI) was calculated as the sum of each FIC and interpreted as follows: FICI≤0.5, ‘synergy’; 0.5<FICI≤4, ‘no interaction’; FICI>4, ‘antagonism’. At least three independent experiments were performed and mean FICI was calculated.

### Antibiofilm Activity

Biofilms are sessile communities that offer protection from external factors such as antimicrobial drugs and, in case of an infection, are particularly relevant after catheterization and stenting. Germ tube formation plays a key role in biofilm formation, but it also facilitates cellular invasion of C. albicans.

In these assays, supra- and sub-MIC concentrations were tested when possible, maintaining DMSO concentrations below 2%. There was significant inhibition of biofilm formation in concentrations ranging between 2×MIC and ¼×MIC (FIG. 12A). There was complete inhibition of dimorphic transition at 64 μg/mL (MIC) and a significant inhibition at 32 and 16 μg/mL (½×MIC and ¼×MIC, respectively) (FIG. 12B).

### Biofilm Formation

The effect of compound 1 on biofilm formation of C. albicans ATCC 10231 was evaluated through quantification of total biomass by crystal violet staining. Briefly, compound 1 in concentrations ranging between 128 and 16 μg/mL (2×MIC and ¼×MIC), was added to yeast suspensions prepared in RPMI, at a final concentration of (1.0±0.2)×10⁶ CFU/mL, as determined by cell counts using a haemocytometer. A control with appropriate concentration of DMSO, as well as a negative control (RPMI alone), were included. Sterile 96-well flat-bottomed untreated polystyrene microtiter plates were used. After a 48 h incubation at 35° C., the biofilms were stained with 1% (v/v) crystal violet for 5 min. The stain was solubilized with 33% (v/v) acetic acid and the biofilm biomass was quantified by measuring the absorbance of each sample at 570 nm in a microplate reader (Thermo Scientific Multiskan® EX, Thermo Fisher Scientific, Waltham, MA, USA). The background absorbance (RPMI without inoculum) was subtracted, and the data are presented as percentage of control. Three independent assays were performed in triplicate for each experimental condition.

### Germ Tube Formation

The effect of compound 1 in germ tube formation of C. albicans ATCC 10231 was determined as previously described. Briefly, cell suspensions were prepared in NYP medium (N-acetylglucosamine [Sigma, St. Louis, MO, USA; 10-3 mol/L], yeast nitrogen base [Difco, New Jersey, USA; 3.35 g/L], proline [Fluka, Buchs, St. Gallen, Switzerland; 10⁻³ mol/L], and NaCl [4.5 g/L], pH 6.7±0.1) and adjusted to a density of (1.0±0.2)×10⁶ CFU/mL, as determined by cell counts using a haemocytometer. An appropriate volume of compound stock solution at 10 mg/mL was added to obtain final concentrations ranging between 64 and 8 μg/mL. Filamentation controls were included in each assay with and without 0.64% DMSO. Following a 3 h incubation at 37° C., 100 cells from each sample were counted, using a haemocytometer, and the percentage of germ tubes was determined. Three independent assays were performed.

The subject matter described above is provided as an illustration of the present invention and, therefore, should not be construed to limit it. The terminology employed for the purpose of describing preferred embodiments of the present invention should not be restricted to them.

As used in the description, defined and indefinite articles, in their singular form, are intended for interpretation to also include plural forms, unless the context of the description explicitly indicates otherwise.

Undefined articles “one” should generally be interpreted as “one or more”, unless the meaning of a singular modality is clearly defined in a specific situation.

It will be understood that the terms “understand” and “include”, when used in this description, specify the presence of characteristics, elements, components, steps and related operations, but do not exclude the possibility of other characteristics, elements, components, steps and operations as well contemplated.

As used throughout this patent application, the term “or” is used in an inclusive sense rather than an exclusive sense, unless the exclusive meaning is clearly defined in a specific situation. In this context, a phrase of the type “X uses A or B” should be interpreted as including all relevant inclusive combinations, for example “X uses A”, “X uses B” and “X uses A and B”.

In addition, the articles “a” and “an” as used in this application and the appended claims should generally be construed to mean “one or more” unless specified otherwise or clear from the context to be directed to a singular form.

The present invention may be embodied in other specific forms without departing from its scope or essential characteristics. The described embodiments are to be considered in all respects only as illustrative and not restrictive.

The scope of the invention is, therefore, indicated by the appended claims rather than by the foregoing description. All changes which come within the meaning and range of equivalency of the claims are to be embraced within their scope.

### CITATION LIST

Here follows the list of citations:

### PATENT LITERATURE

PTL 1: U.S. Pat. No. 5,583,239 A
PTL 2: US2021363174 (A1)
PTL 3: US2017258963 (A1)
PTL 4: US2018272034 (A1)

### Non-Patent Literature

NPL 1: Mukhopadhyay, S.; Maitra, U.; Ira; Krishnamoorthy, G.; Schmidt, J.; Talmon, Y. Structure and Dynamics of a Molecular Hydrogel Derived from a Tripodal Cholamide. J Am Chem Soc 2004, 126, 15905-15914, doi:10.1021/ja046788t.
NPL2. Löfman, M.; Koivukorpi, J.; Noponen, V.; Salo, H.; Sievänen, E. Bile acid alkylamide derivatives as low molecular weight organogelators: systematic gelation studies and qualitative structural analysis of the systems. J Colloid Interface Sci 2011, 360, 633-644, doi:10.1016/j.jcis.2011.04.112.
NPL3. Koivukorpi, J.; Kolehmainen, E. Novel deoxycholic acid alkylamidephenylurea-derived organogelators. Tetrahedron Letters 2010, 51, 1199-1201, doi:https://doi.org/10.1016/j.tetlet.2009.12.101.
NPL 4: Kramer, W.; Wess, G. Bile Acid Derivatives, Processes for their Preparation and Use as Pharmaceuticals. U.S. Pat. No. 5,668,126, 1997.
NPL5. Kikuchi, K.; Bernard, E. M.; Sadownik, A.; Regen, S. L.; Armstrong, D. Antimicrobial activities of squalamine mimics. Antimicrob Agents Chemother 1997, 41, 1433-1438, doi:10.1128/AAC.41.7.1433.
NPL 6: Regen, S. L. Antimicrobial Sterol Conjugates. U.S. Pat. No. 5,583,239, 1996.
NPL7. Schmidt, E. J.; Boswell, J. S.; Walsh, J. P.; Schellenberg, M. M.; Winter, T. W.; Li, C.; Allman, G. W.; Savage, P. B. Activities of cholic acid-derived antimicrobial agents against multidrug-resistant bacteria. J Antimicrob Chemother 2001, 47, 671-674, doi:10.1093/jac/47.5.671.
NPL8. Li, C.; Lewis, M. R.; Gilbert, A. B.; Noel, M. D.; Scoville, D. H.; Allman, G. W.; Savage, P. B. Antimicrobial activities of amine- and guanidine-functionalized cholic acid derivatives. Antimicrob Agents Chemother 1999, 43, 1347-1349, doi:10.1128/AAC.43.6.1347.
NPL9. Stoltz, K. L.; Erickson, R.; Staley, C.; Weingarden, A. R.; Romens, E.; Steer, C. J.; Khoruts, A.; Sadowsky, M. J.; Dosa, P. I. Synthesis and Biological Evaluation of Bile Acid Analogues Inhibitory to Clostridium difficile Spore Germination. J Med Chem 2017, 60, 3451-3471, doi:10.1021/acs.jmedchem.7b00295.
NPL 10: 0. Sorg, J.; Sonenshein, A., L. Methods and Compositions for Inhibiting Clostridium difficile Spore Germination and Outgrowth. WO 2010/062369, 2010.
NPL11. Aher, N. G.; Pore, V. S.; Mishra, N. N.; Shukla, P. K.; Gonnade, R. G. Design and synthesis of bile acid-based amino sterols as antimicrobial agents. Bioorg Med Chem Lett 2009, 19, 5411-5414, doi:10.1016/j.bmcl.2009.07.117.
NPL12. Agarwal, D. S.; Singh, R. P.; Lohitesh, K.; Jha, P. N.; Chowdhury, R.; Sakhuja, R. Synthesis and evaluation of bile acid amides of α-cyanostilbenes as anticancer agents. Molecular Diversity 2018, 22, 305-321, doi:10.1007/s11030-017-9797-9.
NPL13. Mishra, S.; Patel, S. Design, Synthesis, and Anti-bacterial Activity of Novel Deoxycholic Acid-Amino Alcohol Conjugates. Med Chem 2020, 16, 385-391, doi:10.2174/1573406415666190206231002.
NPL 14: 4. Rathbone, D. L.; Thington, T.; Al-Malaika, S.; Hird, M. J.; Quayle, A. R. Compound for Treating Clostridium difficile. WO 2016/083819 A1, 2016.
NPL15. Singla, P.; Dalal, P.; Kaur, M.; Arya, G.; Nimesh, S.; Singh, R.; Salunke, D. B. Bile Acid Oligomers and Their Combination with Antibiotics To Combat Bacterial Infections. J Med Chem 2018, 61, 10265-10275, doi:10.1021/acs.jmedchem.8b01433.
NPL16. Vatmurge, N. S.; Hazra, B. G.; Pore, V. S.; Shirazi, F.; Chavan, P. S.; Deshpande, M. V. Synthesis and antimicrobial activity of β-lactam-bile acid conjugates linked via triazole. Bioorg Med Chem Lett 2008, 18, 2043-2047, doi:https://doi.org/10.1016/j.bmcl.2008.01.102.
NPL17. Neves, A. R.; Almeida, J. R.; Carvalhal, F.; Camara, A.; Pereira, S.; Antunes, J.; Vasconcelos, V.; Pinto, M.; Silva, E. R.; Sousa, E.; et al. Overcoming environmental problems of biocides: Synthetic bile acid derivatives as a sustainable alternative. Ecotoxicology and Environmental Safety 2020, 187, 109812, doi:https://doi.org/10.1016/j.ecoenv.2019.109812.
NPL 18: 8. Costa F., Carvalho I. F., Montelaro R. C., Gomes P., Martins M. C. L. Covalent immobilization of antimicrobial peptides (AMPs) onto biomaterial surfaces. Acta Biomater. 2011, 7, 1431-1440. 10.1016/j.actbio.2010.11.005.
NPL19. Pollard, J. E., J. Snarr, V. Chaudhary, J. D. Jennings, H. Shaw, B. Christiansen, J. Wright, W. Jia, R. E. Bishop and P. B. Savage (2012). “In vitro evaluation of the potential for resistance development to ceragenin CSA-13.” Journal of Antimicrobial Chemotherapy 67(11): 2665-2672.
NPL20. Stiefel, P., S. Schmidt-Emrich, K. Maniura-Weber and Q. Ren (2015). “Critical aspects of using bacterial cell viability assays with the fluorophores SYTO9 and propidium iodide.” BMC Microbiology 15(1): 36.

1.-27. (canceled)

28. A compound of general formula (I) 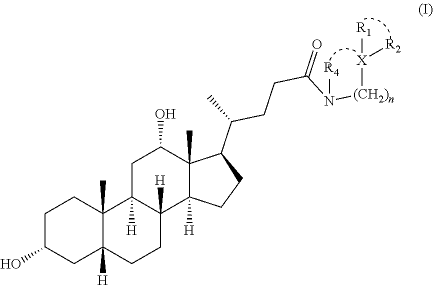
or an acceptable salt, a hydrate, a solvate, an enantiomer, an atropisomer, a polymorph or an ester thereof
characterized by the fact that:
X is an atom selected from the group consisting of N, O or C, with the proviso that X is bonded to at least one of R₁ or R₂ when X is N or C, and X is optionally bonded to R₁ or R₂ when X is O;
each of R₁ or R₂ are independently selected from the group consisting of H, a C₁-C₈ alkyl group, a C₆-C₁₂ aryl group, a 3-12-membered heterocyclyl ring, a 5-12 membered heteroaryl ring, —NH—COOR₃ wherein R₃ is H or a C₁-C₈ alkyl group, —CO—NH₂, —NHCOCF₃, —NH-Aryl, —NH—C(Aryl)₃, —N═CH-Aryl, —NH—S(O)₂—
Aryl wherein Aryl is benzyl or toluyl; a —COO-alkyl group, —NH₂, —NH—, 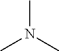
—N═, or 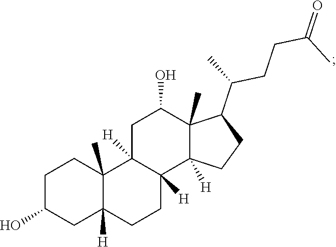 [SMILES: CC(=O)CC[C@@H](C)[C@H]1CC[C@H]2[C@@H]3CC[C@@H]4C[C@H](O)CC[C@]4(C)[C@H]3C[C@H](O)[C@]12C]
with the proviso that when R₁ or R₂ is —NH—, 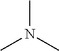
or —N═, X is taken together with the R₁ and R₂ of the compound of general formula (I) to form a 3-12-membered heterocyclyl or condensed heterocyclyl ring or 5-12 membered heteroaryl or condensed heteroaryl ring, provided that the rules of valency permit, wherein each heterocyclyl or heteroaryl ring optionally contains at least one additional heteroatom selected from the group consisting of O and N;
R₄ is selected from the group consisting of H, a single bond or a double bond;
with the proviso when R₄ is a single bond or a double bond, R₄ is taken together with X to form a 3-12-membered heterocyclyl ring or 5-12 membered heteroaryl ring;
n is an integer in the range from 1 to 6.

29. The compound according to claim 28 wherein
X is N or C, wherein X is bonded to R₁ and R₂;
each of R₁ or R₂ are independently selected from the group consisting of H, a C₁-C₈ alkyl group, a C₆-C₁₂ aryl group or a 5-12 membered heteroaryl ring,
the C₁-C₈ alkyl group being ethyl,
the C₆-C₁₂ aryl group being phenyl,
the 5-12 membered heteroaryl ring being benzoimidazolyl,
R₄ is H; and
n is an integer in the range from 2 to 4.

30. The compound according to claim 28 wherein
X is an atom selected from the group consisting of N, O or C, with the proviso that:
when X is N, R₄ is a single bond; and R₄ is taken together with X to form a piperidyl or a piperazinyl, which are optionally substituted by a —COO—C₁-C₈ alkyl; and
when X is O, R₄ is a single bond; and R₄ is taken together with X to form a morpholinyl; and
when X is C, X is bonded to R₁ and R₂, which are H; and R₄ is a single bond;
and R₄ is taken together with X to form a piperidyl,
n is an integer in the range from 2 to 4.

31. The compound according to claim 28 wherein
X is N and is bonded to R₁ and R₂;
each of R₁ or R₂ are independently selected from the group consisting of H, a C₁-C₈ alkyl group, or 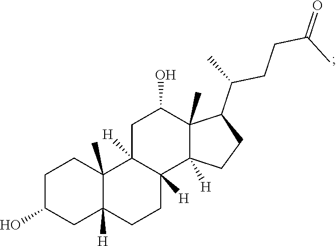 [SMILES: CC(=O)CC[C@@H](C)[C@H]1CC[C@H]2[C@@H]3CC[C@@H]4C[C@H](O)CC[C@]4(C)[C@H]3C[C@H](O)[C@]12C]
R₄ is a single bond; and R₄ is taken together with X to form a piperidyl or a piperazinyl; and
n is an integer in the range from 2 to 4.

32. The compound according to claim 28 wherein the compound is selected from the group consisting of

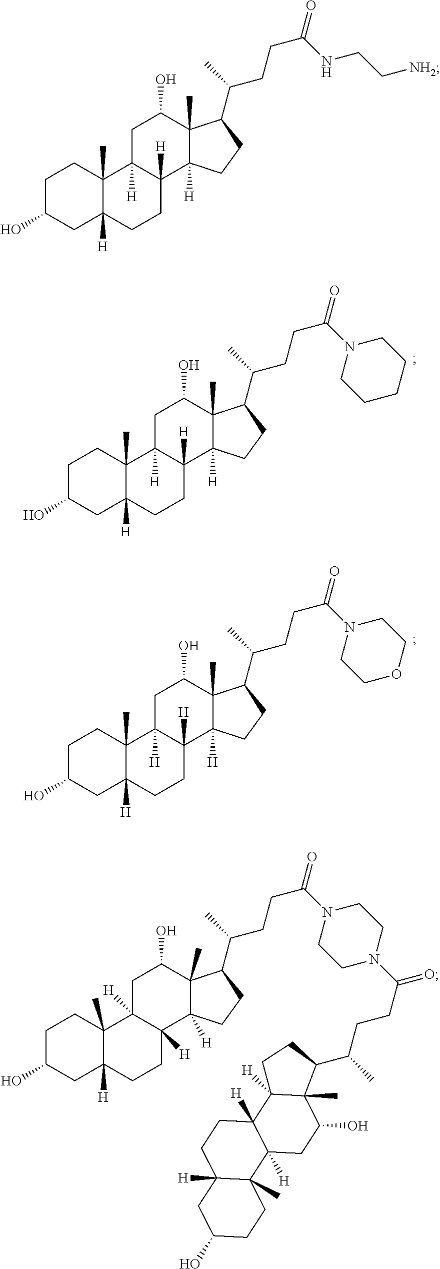

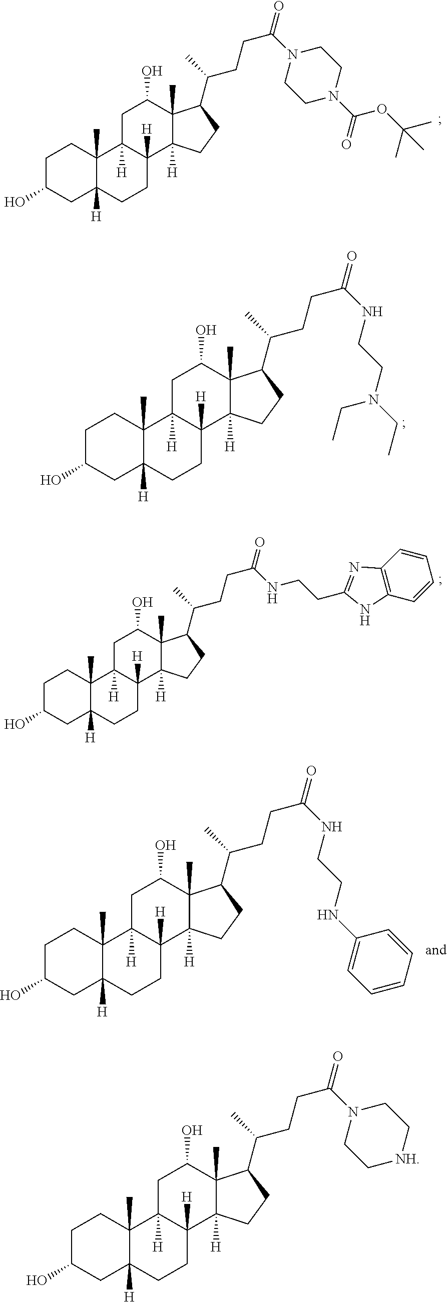

33. The compound according to claim 28, wherein the salt of the compound is a fluoride, chloride, bromide, iodide, acetate, citrate, maleate, or mesylate.

34. Method for obtaining the compound as defined in claim 28 characterized by the coupling of an amine and a carboxylic acid of formula (II) 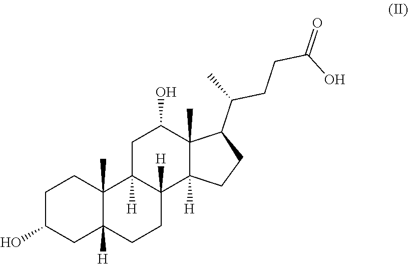
using (1-cyano-2-ethoxy-2-oxoethylidenaminooxy) dimethylaminomorpholino-carbenium hexafluorophosphate (COMU); wherein the amine is N,N-diisopropylethylamine.

35. Method for obtaining the compound as defined in claim 28 characterized by the deprotection of an amide of formula (III) 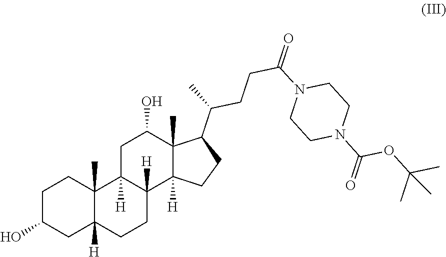
with trifluoroacetic acid.

36. Compound as defined in claim 28 characterized by the fact that it is for use as a medicament in humans or animals to treat or prevent bacterial or fungal infections.

37. Compound according to claim 36 wherein the bacterial infection is caused by a Gram-positive bacteria selected from the group consisting of Staphylococcus spp., Enterococcus spp. and Salmonella spp. or a Gram-negative bacteria selected from the group consisting of Acinetobacter spp., Listonella spp, Yersinia spp., Tenacibaculum spp., Campylobacter spp., Klebsiella spp. and Pseudomonas spp.

38. Compound according to claim 36 wherein the fungal infection is caused by a yeast or a filamentous fungi selected from the group consisting of Candida spp., Cryptococcus spp., Aspergillus spp., Fusarium spp., Mucor spp.b, Lichtheimia spp.b, Scedosporium spp., Trichophyton spp., Microsporum spp., Nannizzia spp. and Saprolegnia spp.

39. Compound as defined in claim 28 characterized by the fact that it is for medical use for antibiofilm purposes in medical devices.

40. Composition comprising the compound described in claim 28 and a pharmaceutically acceptable excipient, wherein the composition is a polymeric matrix composition or a coating composition comprising from 0.1 to 10 wt % of the compound.

41. Composition according to claim 40 wherein the polymeric matrix composition is a polydimethylsiloxane (PDMS) based matrix composition.

42. Composition according to claim 40 further comprising an antibiotic selected from the group consisting of fluoroquinolones, macrolides, aminoglycosides, β-lactams, and polypeptides.

43. Composition according to claim 40 further comprising at least one of the additives selected from the group consisting of: a dye, a polymer, a filler, an essential oil, a stabilizer, a surfactant, a crosslinker agent, a curing agent, a biocide, a solvent, or mixtures thereof.

44. Composition as defined in claim 40 characterized by the fact that it is for use as a medicament in humans or animals to treat or prevent bacterial or fungal infections.

45. Composition according to claim 44 wherein the bacterial infection is caused by a Gram-positive bacteria selected from the group consisting of Staphylococcus spp., Enterococcus spp. and Salmonella spp. or a Gram-negative bacteria selected from the group consisting of Acinetobacter spp., Listonella spp, Yersinia spp., Tenacibaculum spp., Campylobacter spp., Klebsiella spp. and Pseudomonas spp.

46. Compound according to claim 44 wherein the fungal infection is caused by a yeast or a filamentous fungi selected from the group consisting of Candida spp., Cryptococcus spp., Aspergillus spp., Fusarium spp., Mucor spp.b, Lichtheimia spp.b, Scedosporium spp., Trichophyton spp., Microsporum spp., Nannizzia spp. and Saprolegnia spp.

47. Composition as defined in claim 40 characterized by the fact that it is for medical use for antibiofilm purposes in medical devices.
